(19) United States
(12) Patent Application Publication
(10) Pub. No.: US 2026/0240573 A1
(43) Pub. Date: Aug. 20, 2026

(54) SURGICAL CORD TENSIONING DEVICES, SYSTEMS, AND METHODS

(71) Applicant: Highridge Medical, LLC, Westminster, CO (US)

(72) Inventors: Randall G. Mast, Denver, CO (US); Curtis Tade, Westminster, CO (US); David Ayers, Westminster, CO (US); Greg Sumida, Westminster, CO (US); John Spohn, Westminster, CO (US); Guillaume Quetier, Lakewood, CO (US); Caleb Lee Stoll, Broomfield, CO (US)

(21) Appl. No.: 19/541,732

(22) Filed: Feb. 17, 2026

Related U.S. Application Data
(60) Provisional application No. 63/758,972, filed on Feb. 14, 2025.

(51) Int. Cl. A61B 17/70; A61B 17/00

(52) U.S. Cl. CPC A61B 17/7083; A61B 2017/00367

(57) ABSTRACT

A system includes a tensioner, a tension indicator, a tensioner extension, and a counter tensioner. The tensioner may include a nose, a reel with cord retention notches and cord alignment grooves configured to hold and tension a cord. The tension indicator may include a spring in a housing having a window with cord tension indications and an aperture for receiving the cord. The counter tensioner may be releasably couplable to a head of an implant at a proximal end thereof via an internal sleeve with a live spring with a pin to engage a mating feature on the head of the implant. The counter tensioner may guide the cord to a port proximate the distal end thereof, the port for receiving nose of the tensioner. The counter tensioner may enable translation of the implant relative to another implant implanted in an adjacent or nearby vertebrae.

### CROSS-REFERENCE TO RELATED APPLICATIONS

The present invention claims the benefit to and priority of U.S. Provisional Application No. 63/758,972, filed February 14, 2026. The entire disclosure of the above application is incorporated herein by reference.

### BACKGROUND

The present disclosure relates to surgical techniques and devices for use in correcting spinal deformities, such as scoliosis. More specifically, this disclosure discusses various vertebral implants for use in securing a flexible elongated member (e.g., tether or cord) between vertebral bodies and techniques for correction of spinal deformities using the disclosed implants.

Dynamic stabilization techniques, such as vertebral body tethering, are used in spinal treatment procedures for juveniles to permit enhanced mobility of the spine while also providing sufficient counter loading of a spinal curvature to effect treatment through bone growth modulation, particularly during times of rapid growth. Such dynamic stabilization systems may include fixed, uniaxial, or polyaxial bone anchors installed in adjacent or nearby vertebrae of the spine, various cord clamping devices, and a flexible cord secured to the bone anchors that may be tensioned between the bone anchors.

Current techniques and implants suffer from various deficiencies or potential failure modes as well as difficulties in implantation among other things. The following disclosure discusses various implants and surgical procedures to address these and other short comings with traditional approaches to spinal tethering.

### SUMMARY

The present inventors have recognized, among other things, that improving ergonomics and ease of use of surgical cord (tether) tensioning devices, including improving access to remote implants, may be desirable. The present inventors have also recognized that decreasing the time to tension and secure the surgical cord in each implant can improve outcomes and decrease surgeon fatigue. The present specification discloses various methods, devices, systems, and embodiments that may include a cord tensioner that can be removably couplable to a coupler disposed on a proximal end of a counter tensioner, wherein the distal end of the counter tensioner can be rigidly, removably coupleable to a head of an implant. Additionally, the present specification discloses a tension indicator that may be couplable between the cord tensioner and the proximal end of a counter tensioner, wherein the cord tensioner can display tension on a surgical cord (tether).

Accordingly, the present disclosure provides for a system for positioning and tensioning the surgical cord (tether) without repeated threading the cord through various surgical ports (incisions). The system may comprise a tensioner, a tension indicator, a tensioner extension, and a counter tensioner. The tensioner can comprise a nose assembly and a wheel with cord lock notch for applying tension to the cord. The nose assembly can comprise a lumen extending therethrough for receiving the cord. The tensioner extension can include one or more elongate extension members that can accommodate the cord through an internal lumen and couple to the nose of the tensioner or alternatively couple to the tension indicator that is couplable to the nose of the tensioner. The counter tensioner can be releasably coupleable to a head of an implant at a distal end thereof and can guide a set screw into the implant to secure the cord. The counter tensioner may also enable translation of the implant relative to another implant implanted in an adjacent or nearby vertebrae.

Further, the counter tensioner can be utilized as described in co-pending Application Serial Number 17/141,657, Titled “SURGICAL CORD TENSIONING DEVICES, SYSTEMS, AND METHOD,” filed January 5, 2021, which is hereby incorporated by reference in its entirety. Further the tensioner, counter tensioner, and tensioner extension may be utilized as described in U.S. Patent Application No. 18/330,595, filed on June 7, 2023; which is a continuation of U.S. Patent Application No. 17/164,447, filed February 1, 2021, now U.S. Patent No. 11,690,656, issued on July 4, 2023; which is a continuation of U.S. Patent Application No. 16/156,158, filed October 10, 2018, now U.S. Patent No. 10,939,941, issued on March 9, 2021; which is a continuation-in-part of U.S. Patent Application No. 16/115,441, filed August 28, 2018, now U.S. Patent No. 10,905,474, issued February 2, 2021; which claims the benefit of priority under 35 U.S.C. § 119(e) of U.S. Provisional Patent Application Number 62/551,379, filed August 29, 2017; all of which are hereby incorporated by reference in their entireties.

In an embodiment, the present disclosure provides for a method that can include the steps of inserting a counter tensioner into a patient and coupling the counter tensioner to an implant; coupling a tension indicator to a port at the proximal end of the counter tensioner; coupling a nose assembly of a tensioner to another port of the tension indicator; guiding a cord through the counter tensioner, tensioner indicator, and tensioner; and securing the cord to a wheel notch of the tensioner therein; tensioning the cord by rotating a wheel on the tensioner to translate the elongate shaft and the cord; and translating the implant relative to an implant in an adjacent or nearby bone.

In another embodiment, the present disclosure discusses a method for positioning and tensioning a surgical cord using a secondary (auxiliary) surgical port and a tensioner extension threaded over the cord. In this embodiment, the cord is initially secured in a first implant with the cord subsequently routed through the body to the secondary surgical port. The cord is then threaded through the tensioner extension, and the tensioner extension is maneuvered into the body adjacent to a second implant. The tensioner extension, in combination with the counter tensioner or other instruments, is used to position the cord in the second implant. Once the cord is in the second implant, cord can be threaded through the tension indicator and tensioner coupled to the proximal end of the tensioner extension, and the instruments can be utilized to tension the cord between the first implant and the second implant. Once tensioned, the cord can be secured with a set screw delivered through a channel of the counter tensioner instrument. Upon completion of securing the cord to the second implant, the tensioner extension can maneuver the cord into a third implant and repeat steps to tension and secure the cord in the third implant. This process can be repeated for all implants being used to correct the spinal deformity.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below. In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an embodiment of a system according to the present disclosure;

FIG. 2 is a cross-sectional view of one embodiment of a tensioner according to the present disclosure;

FIG. 3A is a perspective view of an embodiment of a counter tensioner according to the present disclosure and FIG. 3B is an exploded view of the counter tensioner of FIG. 3A;

FIG. 4A is a partial perspective view of an embodiment of a proximal end of the system in use in a first position and FIG. 4B is a partial perspective view of the distal end of the embodiment of FIG. 4A in use;

FIG. 5A is a partial perspective view of an embodiment of a proximal end of the system in use in a second position and FIG. 5B is a partial perspective view of the distal end of the embodiment of FIG. 5A in use;

FIG. 6 is a flow chart illustrating steps in one exemplary method according to the present disclosure;

FIGS. 7A – 7G illustrate a cord tensioning system, according to various embodiments of the present disclosure;

FIG. 8A is a drawing illustrating a cord tensioning system operating through an auxiliary surgical port, according to various embodiments of the present disclosure;

FIG. 8B is a drawing illustrating a cord tensioning instrument including a tensioner extension and secondary extension, according to various embodiments of the present disclosure;

FIGS. 9A-9E are drawings illustrating various aspects of a tensioner extension, according to various embodiments of the present disclosure;

FIGS. 10A-10F are drawings illustrating various aspects of a secondary tensioner extension, according to various embodiments of the present disclosure;

FIGS. 11A-11B are drawings illustrating a connection between a tensioner extension and a secondary tensioner extension, according to various embodiments of the present disclosure;

FIG. 12 is a flowchart illustrating a surgical procedure for tensioning a surgical cord utilizing a tensioner, a tensioner extension, and optionally a secondary extension, according to various embodiments of the present disclosure;

FIGS. 13A-13D are perspective views of a cord tensioning system according to various embodiments of the present disclosure;

FIGS. 14A-14E illustrate various aspects of a counter tensioner according to the present disclosure;

FIGS. 15A-15G are drawings illustrating various aspects of a counter tensioner according to the present disclosure;

FIG. 16A illustrates a perspective view of an embodiment of a cord tensioner according to the present disclosure, FIG. 16B illustrates an exploded perspective view of the cord tensioner of FIG. 16A, and FIGS. 16C-16F illustrate exploded partial perspective views of different aspects of the cord tensioner of FIG. 16A;

FIGS. 17A-17B illustrate partial exploded views of the cord tensioner of FIG. 16A from a bottom perspective;

FIG. 18A illustrates a top view of the cord tensioner of FIG. 16A, FIG. 18B illustrates a partial side view of the cord tensioner of FIG. 16A, and FIGS. 18C-18D illustrate partial views of the cord tensioner of FIG. 16A with a cord according to an embodiment of the present disclosure;

FIGS. 19A-19B illustrate a tensioner and a tension indicator with a cord according to an embodiment of the present disclosure;

FIGS. 20A-20D illustrate a tension indicator according to various aspects of the present disclosure;

FIG. 21 is a flowchart illustrates steps of one exemplary method according to the present disclosure; and

FIG. 22 is a flowchart illustrating a surgical procedure for tensioning a surgical cord utilizing a tensioner, a tension indicator and a counter tensioner according to various embodiments of the present disclosure.

### DETAILED DESCRIPTION

Exemplary embodiments will now be described more fully with reference to the accompanying drawings. Within the following disclosure the terms proximal and distal are generally used with reference to the surgeon using the instruments, rather than with respect to the patient. However, in description of certain examples the use of the terms may be inverted to correspond to the patient point of view.

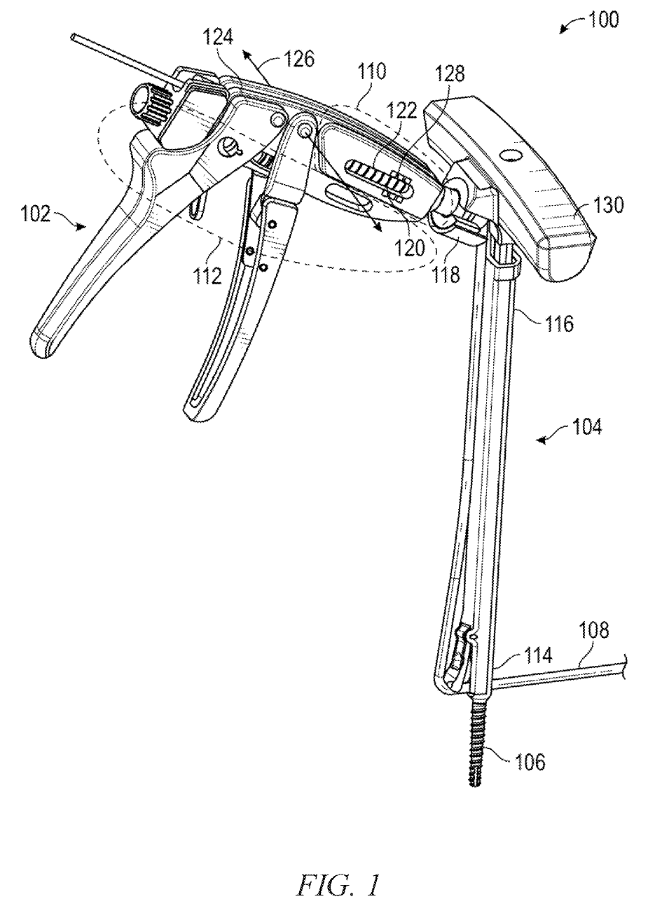

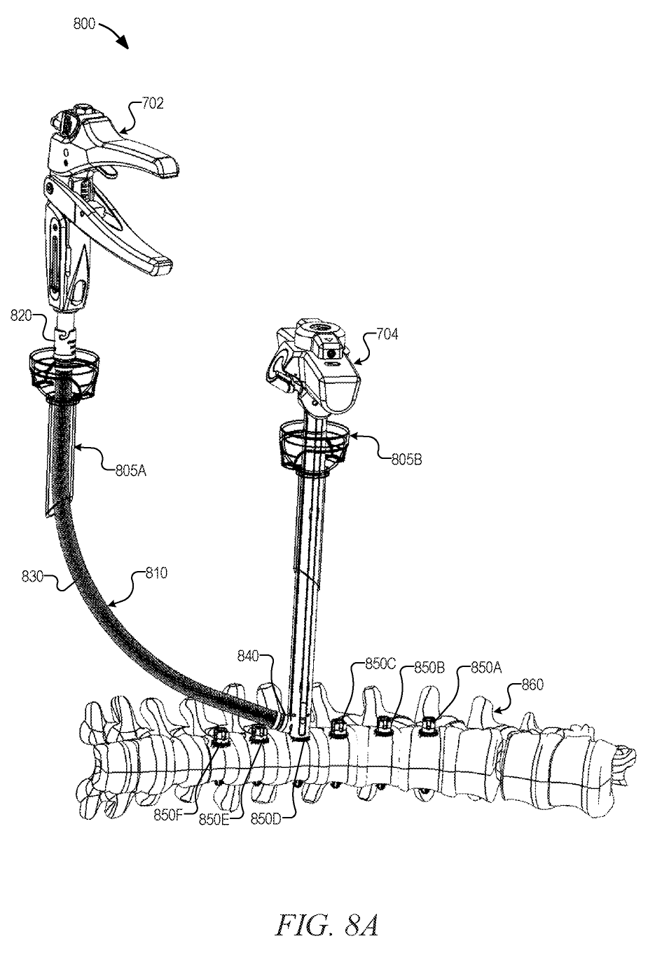

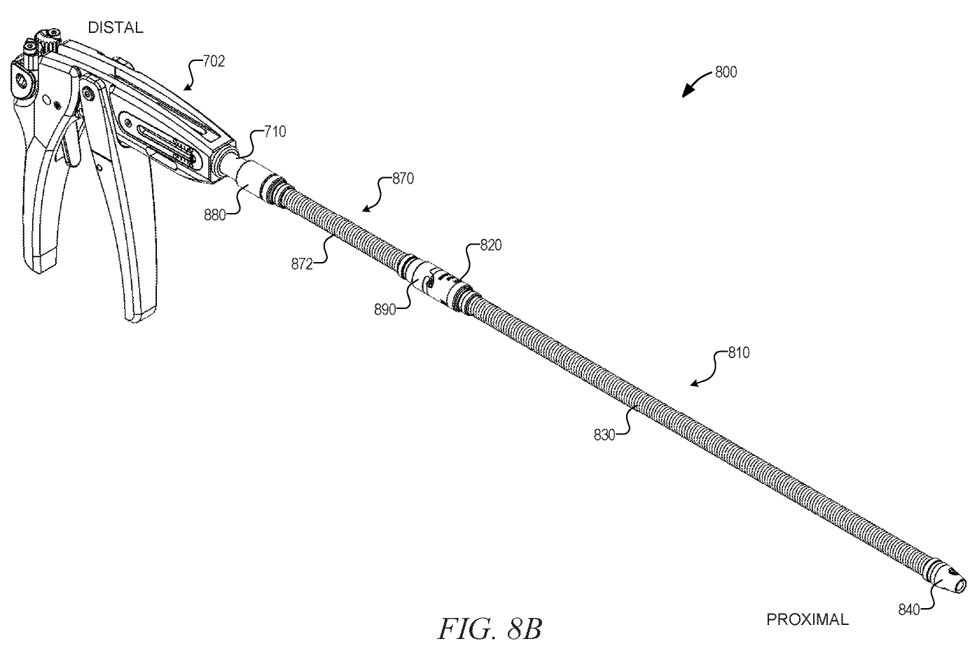

The initial portion of the present disclosure provides for a system comprising a cord tensioner that can be rigidly, removably coupleable to a port disposed on a proximal end of a counter tensioner, wherein the distal end of the counter tensioner can be rigidly, removably coupleable to a head of an implant. Such systems and methods of using the system can improve ergonomics and ease of use by, e.g., enabling one handed operation of a tensioner. Such systems can also increase cord travel per actuation cycle of the cord tensioner and provide a visual indication of cord load during cord tensioning. FIGS. 1-7G address the system comprising a cord tensioner and a counter tensioner according to an embodiment of the disclosure. FIGS. 8A-12 of the disclosure discusses a system utilizing a tensioner extension to facilitate a method of securing a surgical cord (tether) between implants to correct a spinal deformity. The remaining portion of the disclosure discusses a system utilizing a wheel tensioner, a tension indicator, and a counter tensioner according to additional aspect and improvements discussed in the disclosure to facilitate a method of securing a surgical cord (tether) between implants to correct a spinal deformity.

One exemplary embodiment of a system 100 according to the present disclosure is illustrated in FIGS. 1-3. The system 100 can comprise a tensioner 102 and a counter tensioner 104, which are used in combination for manipulating implants 106 coupleable by a cord 108. The tensioner 102 is designed to enable one-handed operation to apply tension to a cord 108. The tensioner 102 can comprise a nose assembly 110 for receiving the cord 108 and a cord lock assembly 112 for securing and applying tension to the cord 108. The counter tensioner 104 can be releasably coupleable to a head of an implant 106 at a distal end 114 thereof and can have a port 118 disposed in a proximal end 116 thereof. The port 118 can be releasably coupleable the nose assembly 110 of the tensioner 102. The counter tensioner 104 can further be rigidly coupleable to both the head of the implant 106 and the nose assembly 110, allowing a surgeon to tension the cord 108 via the tensioner 102 and translate the implant 106 and underlying vertebrae relative to another implant implanted in an adjacent or nearby vertebrae via a handle 130 in the same surgical step and as explained in greater detail below.

The implant 106 can be, for example and without limitation, one or more of, a threaded fastener, a bone screw, a pedicle screw, a staple, a bone clamp, a universal bone clamp, cam blocks, bone plates, and the like. Furthermore, it is contemplated that the tensioner can be used to tension a cord between bones, clamps, or any implant secured to a bone in any manner. The implant 106 can comprise a shaft that purchases underlying bone and a head including features for securely engaging an elongate member that, prior to being secured, can be tensioned to apply forces to manipulate the position of adjacent or nearby vertebrae as a juvenile patient experiences growth. The cord can be, for example and without limitation, one or more of, including optional and various combinations of, any elongate member including a cord, a tether, strap, a cable, a wire, a suture, a thread, or similar flexible ligature. In additional or alternative embodiments, the elongate member can have some beneficial temporal elastic properties that could, for example and without limitation, avoid overcorrection or under correction over the life of an implanted system, and the like. The term “cord” is used throughout the present disclosure, but should be understood to include any elongate member.

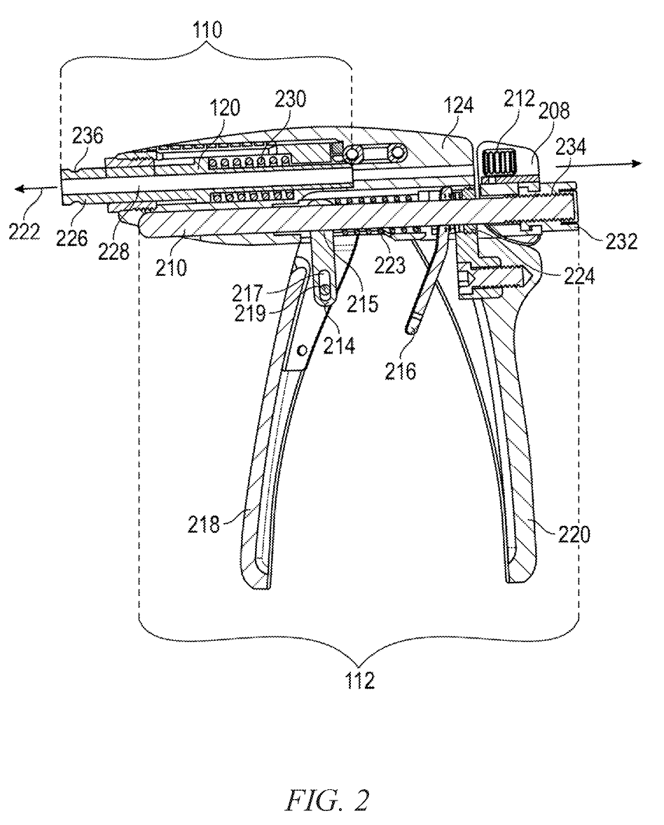

One exemplary embodiment of a tensioner 102 according to the present disclosure is illustrated in FIGS. 1-2. The tensioner 102 can comprise a nose assembly 110 and a cord lock assembly 112 for applying tension to the cord 108. The nose assembly 110 and the cord lock assembly 112 can be at least partially disposed in the main body 124 of the tensioner 102

The cord lock assembly 112 can comprise a cord lock housing 208 disposed at a distal end of an elongate shaft 210 and can engage a cord 108 therein. The cord lock housing 208 can comprise, for example and without limitation, a cam cleat 212 or the like for engaging and disengaging the cord 108. The elongate shaft 210 can be operably coupled to a shaft clutch 214 to drive the elongate shaft 210 proximally and a shaft lock 216 that can resist return of the elongate shaft 210 in the distal direction at the end point of an actuation cycle of the tensioner 102. The shaft clutch 214 can extend from a first end to a second end. The first end can have an opening 215 disposed therein for receiving the elongate shaft 210 and the second end has a slot 217 disposed therein that is transverse to a longitudinal central axis of the elongate shaft 210. The slot 217 receives a pin 219 extending from a front handle 218. Actuation of the front handle 218 shifts the opening 215 disposed in the first end of the shaft clutch 214 off angle to engage and distally translate the elongate shaft 210 as the front handle 218 is actuated towards a stationary rear handle 220. The front handle 218 can be rotatable about a pivot axis 126 (shown in FIG. 1) that is transverse to a longitudinal axis 222 extending from a proximal end to a distal end of the main body.

A shaft spring 223 positioned proximal to the shaft clutch 214 and about the elongate shaft 210 can urge the shaft clutch 214 and front handle 218 back to the starting point of the actuation cycle of the tensioner 102. The shaft lock 216 can be biased via a distally-located lock spring 224 to allow distal translation but not distal translation of the elongate shaft 210. The shaft lock 216 can be released by articulating the shaft lock 216 proximally if the tension on the cord 108 needs to be released, such as once the cord 108 is secured within the implant 106. Additionally, or alternatively, a cord nut 232 disposed on proximate a proximal end of the cord lock housing 108 can be rotated to adjust the cord tension via engagement with threading 234 disposed on the proximal end of the elongate shaft 210. In one example, the elongate shaft 210 can be proximally translated from about 30mm to about 35mm during each actuation cycle or stroke of the shaft clutch 214. The tensioner 102 can be actuated via a single hand of a surgeon, freeing the other hand for performing other surgical tasks during and adjacent to cord tensioning.

The nose assembly 110 can comprise a piston 226 having a lumen 228 extending therethrough for receiving the cord and a load spring 230 positionable in contact with an indicator region 120 disposed on the piston 226. The indicator region 120 of the piston 226 can have a cross-sectional diameter than can be greater than or equal to any other portion of the piston 226. A nose cap 232 can retain the piston 226 and load spring 230 in the main body 124 of the tensioner 102. The load spring 230 can be calibrated so that the position of the indicator region 120 of the piston 226 correlates to the load applied to the cord as the cord 108 is tensioned and there is a counter pressure applied by the counter tensioner 104 to the nose assembly 110. The indicator region 120 can be visible through a window 122 disposed on the main body 124 of the tensioner 102 having indicia 128 printed adjacent to thereto indicative of cord load at various indicator region positions relative to the tensioner main body 124, as best illustrated in FIG. 1. The piston 226 can have a detent 236 disposed in a circumferential outer surface of the piston 226 proximate a distal end thereof for coupling to the counter tensioner 104.

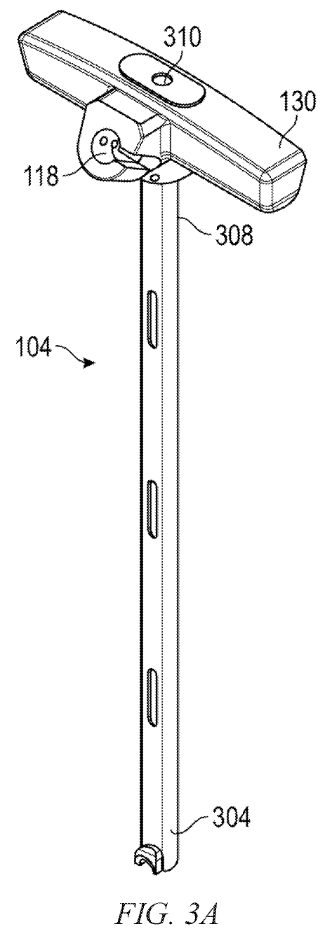

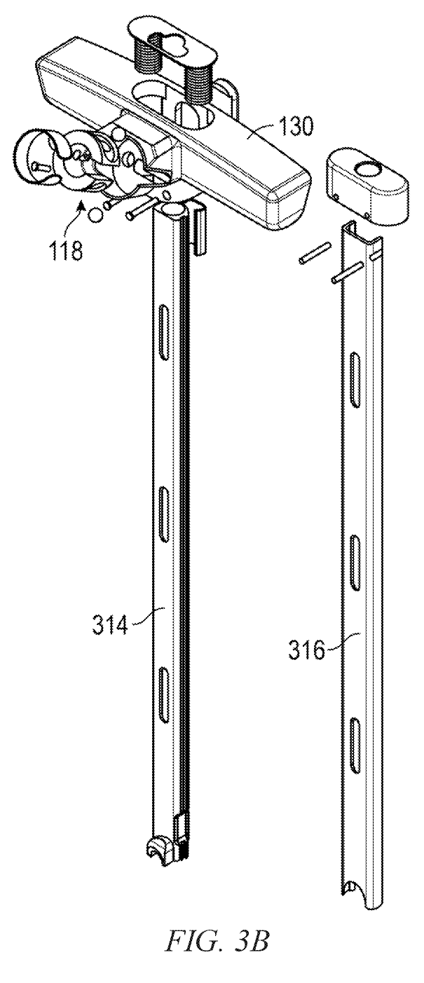

One exemplary embodiment of a counter tensioner 104 according to the present disclosure is illustrated in FIGS. 1, 3A, and 3B. The counter tensioner 104 can be releasably coupleable to the head of an implant 106 at a distal end 304 thereof. The counter tensioner 104 can guide the cord 108 to a port 118 proximate to the proximal end 308 thereof. The port 118 can allow the cord to be fed through the nose assembly 110 to be secured in the cord lock housing 206 of the cord lock assembly 112. The port 118 can rigidly, releasably receive the nose assembly 110 of the tensioner 102 via a capture ball disposed in the port 118 for engaging the detent 236 of the piston 226. The counter tensioner 104 can further comprise a lumen 310 disposed therein extending from the distal end 304 to the proximal end 308 thereof. The port 118 can be disposed at an angle that is not parallel to a longitudinal axis of the lumen 310. A set screw (not shown) can be deliverable through the lumen 310 and engageable with the head of the implant to secure the cord therein with a driver (not shown). The counter tensioner 104 can comprise a handle 130 that can facilitate translation of the implant 106 relative to another implant implanted in an adjacent or nearby vertebrae. Accordingly, vertebral translation, cord tensioning, and cord fixation can be performed in the same surgical step.

The counter tensioner 104 can further comprise an inner sleeve 314 and an outer sleeve 316. The inner sleeve 314 can comprise a plurality of outwardly biased arms at a distal end thereof that are engageable with cooperating features disposed on the head of the implant. Advancing the outer sleeve 316 distally over the inner sleeve 314 and at least partially over the head of the implant can urge the outwardly biased arms into secure engagement with cooperating features on the head of the implant.

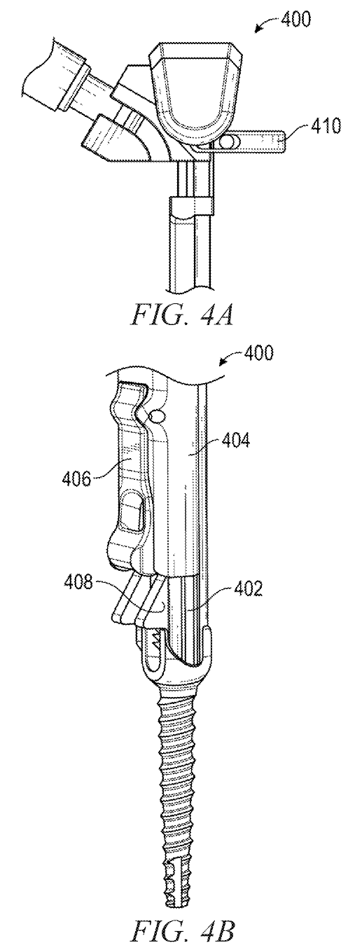

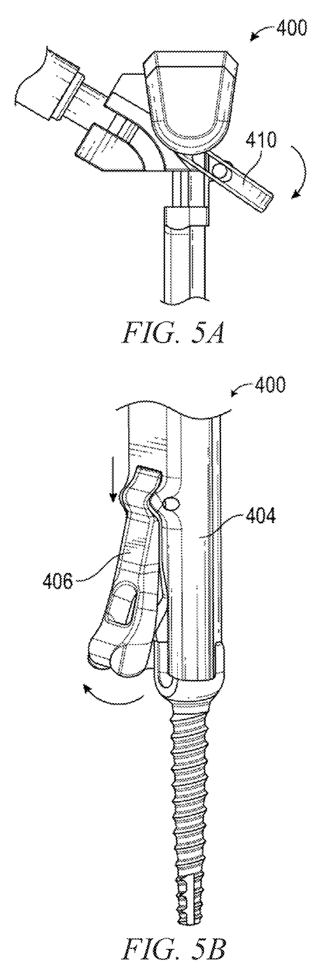

Another exemplary embodiment of a counter tensioner 400 according to the present disclosure is illustrated in FIGS. 4A-5B. Here, the counter tensioner 400 can comprise an inner sleeve 402 and an outer sleeve 404. A distal end of the outer sleeve 404 comprises a fulcrum 406 that can move from a first position where the fulcrum 406 is parallel to a longitudinal axis of the counter tensioner 400 to a second position where the fulcrum 406 pivots outwardly from the longitudinal axis of the counter tensioner 400. The inner sleeve 402 can comprise at least one projection 408 that can urge the fulcrum 406 outward as the outer sleeve 404 passes distally over the inner sleeve 402 and at least partially over the head of the implant. The fulcrum 406 serves to change the cord angle, easing local tension on the cord as it is routed up to the tensioner 102 engaged in the port 118. Optionally, a pivoting latch 410 can be provided at a proximal end of the inner sleeve 402. The pivoting latch 410 can be operatively coupled to the counter tensioner 400 so that the pivoting latch 410 moves downward as the fulcrum moves toward a parallel orientation relative to the longitudinal axis of the counter tensioner 400 and upward as the fulcrum pivots outwardly from the longitudinal axis of the counter tensioner. As a skilled artisan will appreciate in light of the present disclosure, the fulcrum can reduce stress applied to the cord during the tensioning process.

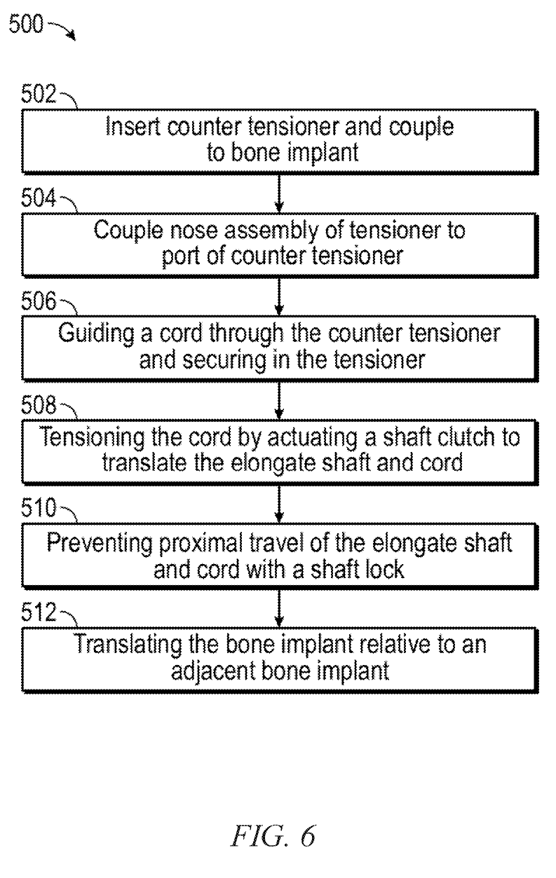

FIG. 6 is a flowchart illustrating a method 500 according to an exemplary embodiment. The method 500 can include operations such as coupling a counter tensioner to an implant at 502; coupling a tensioner to the counter tensioner at 504; routing a cord through the counter tensioner and securing the cord in the tensioner at 506; and tensioning the cord at 508. The method 500 can begin at 502 with a counter tensioner, such as counter tensioner 104, being inserted through an incision and coupled to a head of an implant. In an example, the counter tensioner 104 includes an inner sleeve (e.g., inner sleeve 314) including biased arms located at a distal end thereof and an outer sleeve (e.g., outer sleeve 316). In this example, the biased arms can be engaged with cooperating features on the head of the implant, and the outer sleeve can subsequently be slid distally to lock the biased arms onto the head of the implant. In another example, the counter tensioner can include a threaded feature, a quick connect feature, or the like at a distal end thereof to couple the counter tensioner 104 to complementary features disposed on the head of the implant. In yet another additional or alternative example, the counter tensioner 104 can engage the head of the implant such that the longitudinal axis of the implant and the longitudinal axis of the counter tensioner are not parallel and the cord can couple the counter tensioner to the implant.

At 504, the method 500 can continue with the tensioner 102 being coupled to the counter tensioner 104. In an example, the tensioner 102 includes a nose assembly 110 including a piston 226 formed from a cylinder with a detent 236 disposed in a circumferential outer surface and proximate a distal end thereof. The detent 236 in the piston 226 can engage a capture ball biased into the port 118.

At 506, the method 500 can continue with guiding a cord from the proximal end of the counter tensioner 104, through the port 118 and a lumen 228 disposed in a piston 226 of the nose assembly 110 of the tensioner 102, and securing the cord within a cord lock housing 208 of a cord lock assembly 112 disposed at the end of an elongate shaft 210, the cord being secured in an implant 106 in an adjacent or nearby vertebrae.

At 508, the method 500 can continue with tensioning the cord 108 by actuating a front handle 218 of the tensioner 102 to cause a shaft clutch 214 to engage and distally translate the elongate shaft 210 and the cord 108.

At 510, the method 500 can continue by preventing proximal travel of the elongate shaft 210 at the end of the stroke of the shaft clutch 214 by causing the shaft lock 216 to engage and prevent return of the elongate shaft 210.

At 512, the method 500 can optionally include translating the implant and underlying vertebrae relative to an implant of an adjacent or nearby vertebrae. Here, a surgeon can grasp handle 130 to translate the implant coupled to the counter tensioner 104. The steps of tensioning the cord and translating the vertebrae can be performed simultaneously.

The method can further comprise indicating a load on the cord by the position of an indicator region of a piston of the nose assembly relative to indicia printed adjacent a window in a main body of the tensioner. As the cord is tensioned and counter pressure is applied to the piston of the nose assembly, the piston depressed the load spring and an indicator region of the piston moves in the window disposed in the main body relative to the printed indicia printed adjacent to the window to indicate the load applied to the cord.

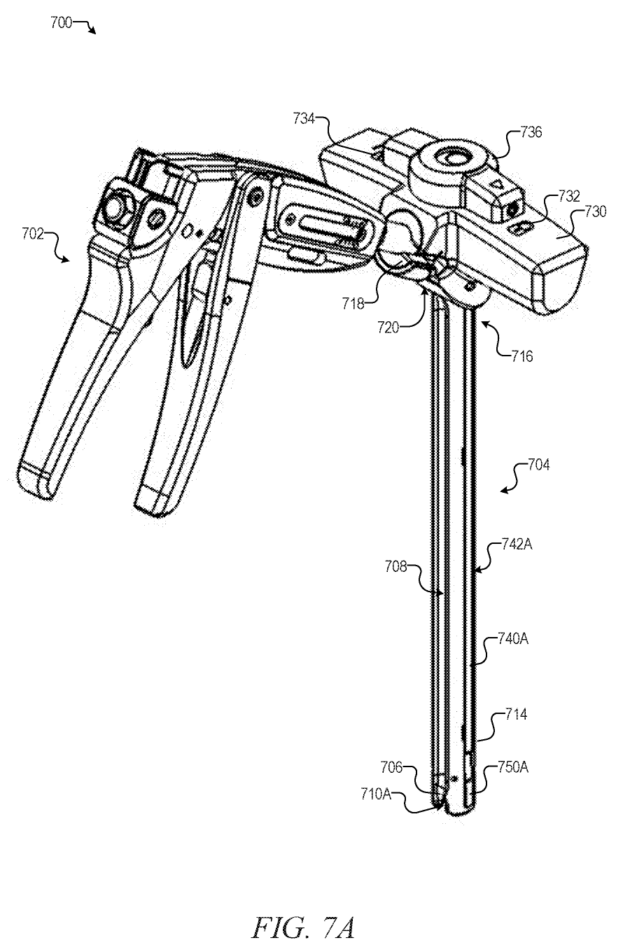

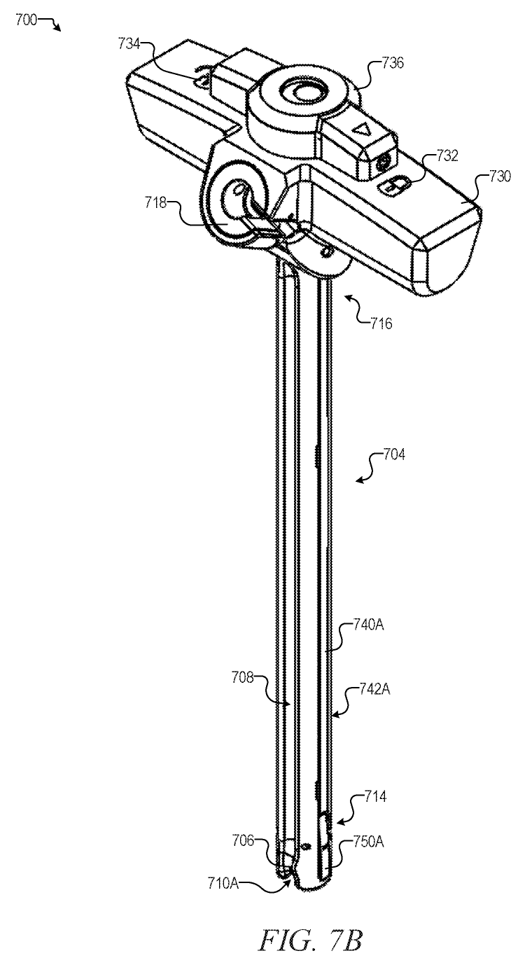

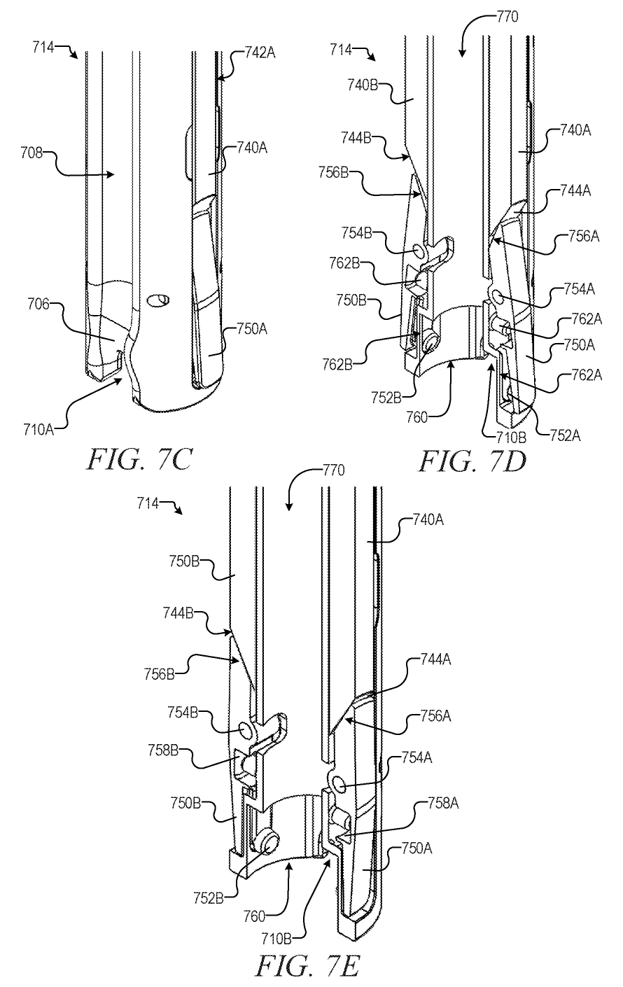

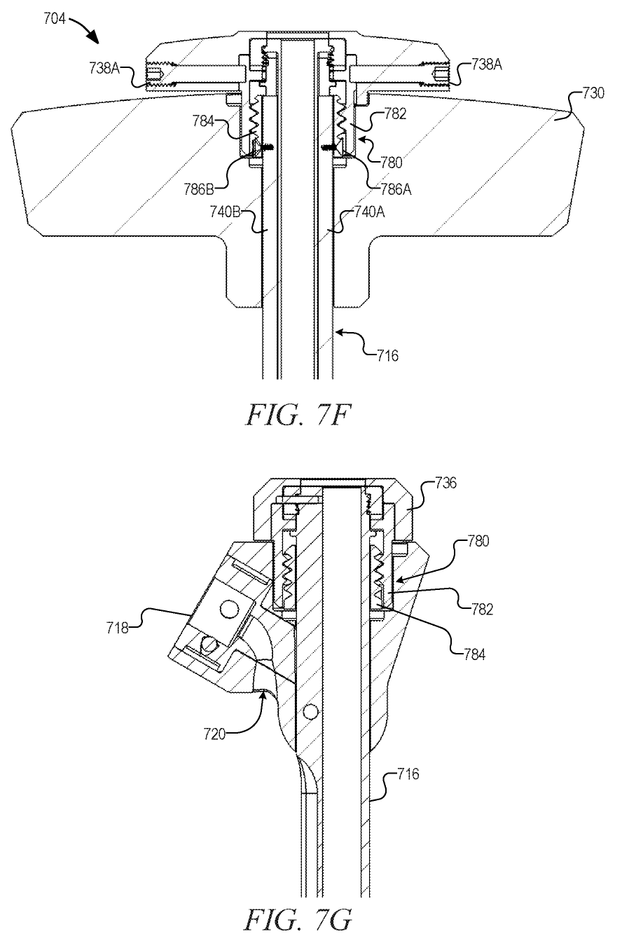

Another exemplary embodiment of a cord tensioning system, system 700, is illustrated in FIGS. 7A-7G. The system 700 can comprise a tensioner 702 and a counter tensioner 704, which are used in combination for manipulating implants 106 coupleable by a cord 108 (not illustrated in conjunction with this example). The tensioner 702 is designed to enable one-handed operation to apply tension to a cord 108 (similar to that shown in FIG. 1). The tensioner 702 includes similar structural features to tensioner 102 discussed in reference to FIGS. 1-3, and will not be further described in reference to this example. The counter tensioner 704 can be releasably coupleable to a head of an implant 106 at a distal end 714 thereof and can have a port 718 disposed in a proximal end 716 thereof. The port 718 can releasably receive the nose assembly of the tensioner 702 (tensioner 102 is interchangeable with tensioner 702). The counter tensioner 704 can further be rigidly coupleable to both the head of the implant 106 and the nose assembly, allowing a surgeon to tension the cord 108 via the tensioner 702 and translate the implant 106 and underlying vertebrae relative to another implant implanted in an adjacent or nearby vertebrae via a handle 730 in the same surgical step and as explained in greater detail below. The remaining discussion of counter tensioner 704 focuses on the aspects that differ from examples discussed above.

In this example, the counter tensioner 704 includes a handle 730 with additional features, such as a lock position 732, an unlock position 734, and a lock handle 736. The lock handle 736 is rotatable between the lock position 732 and the unlock position 734. In the unlock position 734, the counter tensioner 704 can be inserted over an implant, such as implant 106 (e.g., a pedicle screw). Once inserted over the pedicle screw, the lock handle 736 can be rotated into the lock position 732 and elements discussed below will lock the counter tensioner 704 to the pedicle screw. Internal workings of the lock mechanism are discussed in reference to FIGS. 7F and 7G below.

Along the length of the counter tensioner 704 extend a pair of locking extensions 740A, 740B within a pair of locking extension channels 742A, 742B. FIGS. 7A-7B only illustrate locking extension 740A and locking extension channel 742A. In this example, the locking extensions 740A, 740B are square elongate members connected at a proximal end to a locking mechanism operated by the lock handle 736. In other examples, the locking extensions 740A, 740B can be cylindrical rods or other cross-sectional profiles. The locking extension channels in this example are open square (or rectangular) channels, designed to fit the locking extensions 740A, 740B. Moving to the distal end 714 of the counter tensioner 704, the locking extension 740A engages one of the pivot locks 750A (pivot lock 750B is disposed on the opposite side, and shown in FIGS. 7D and 7E). In other examples, the locking extension channels can be a different cross-sectional shape and/or be internal to the counter tensioner 704. FIGS. 7A and 7B also illustrate a cord fulcrum 706, a cord channel 708, and a cord guide 710A (cord guide 710B is an opening on the opposing side of the distal end 714 not illustrated in this example). The cord guide 710A allows the cord to exit the counter tensioner 704 and pedicle screw. The cord guides 710A, 710B are designed to complement the U-shaped pedicle screw head adapted to receive the cord. The cord fulcrum 706 includes an enlarged radius area to smooth directional change in the cord from a first direction essentially transverse to a longitudinal axis of the counter tensioner 704 to a second direction essentially parallel the longitudinal axis (see FIG. 1, the cord fulcrum 706 operates in a manner similar to fulcrum 106). In contrast to fulcrum 106, the cord fulcrum 706 is an integral part of the distal end 714, and does not include any moving parts (e.g., does not pivot). From the cord fulcrum 706, the cord extents proximally along the cord channel 708 until entering the cord conduit 720 at the base of the tensioner port 718, which is more clearly illustrated in FIG. 7G. The cord channel 708, in this example, is a semi-circular recess in the elongate portion of the counter tensioner 704.

FIG. 7C is a close-up perspective view of the distal end 714 of the counter tensioner 704. A number of the structures discussed above are shown in additional detail, including the cord fulcrum 706, the cord channel 708, the cord guide 710A, a distal end of the locking extension 740A, the locking extension channel 742A, and the pivot lock 750A. FIG. 7C illustrates the counter tensioner 704 in an unlocked position. The cord fulcrum 706 in particular is shown in greater detail, in this figure it is easier to make out the radius created by the cord fulcrum 706. The enlarged radius provides a smoother transition for the cord coming out of the cord guide 710A and proceeding up along the cord channel 708.

FIGS. 7D and 7E provide cross sectional views of the distal end 714 of the counter tensioner 704 in an unlocked and locked state, respectively. The cross sections provide a detailed view of both sides of the counter tensioner 704. For example, both locking extensions 740A, 740B are shown extending down opposite sides of the counter tensioner 704 and terminating in extension wedges 744A, 744B. The extension wedges 744A, 744B tapper the locking extensions 740A, 704B down to a thin edge along the distal most portion. In this example, the extension wedges 744A, 744B tapper at approximately 60 degrees, but other tappers can be used. The extension wedges 744A, 744B engage with lock wedges 756A, 756B, respectively. The lock wedges 756A, 756B in this example tapper at a corresponding amount to produce an essentially opposing structure to the extension wedges 74A 744B. In some examples, the ramp (tapper) angle on the lock wedges 756A, 756B is slightly greater or slightly less than the extension wedges 756A, 756B to reduce friction between the surfaces. The locking mechanism within the handle 730 operates to linearly translate the locking extensions 740A, 740B from the unlocked position shown in FIG. 7D to a locked position shown in FIG. 7E. Upon translation, the extension wedges 744A, 744B engage the lock wedges 756A, 756B and cause the pivot locks 750A, 750B to rotate about pivots 754A, 754B to shift locking pins 752A, 752B into recesses within a head of the pedicle screw. The pivot locks 750A, 750B are biased into an unlocked position by springs (not shown for clarity) disposed within spring recesses 758A, 758B with the springs held in place by spring pins 764A, 764B. The locking pins 752A, 752B operate to secure the counter tensioner 704 on the pedicle screw head when pivoted into the locked position (FIG. 7E).

In this example, the locking pins 752A, 752B are cylindrical posts extending radially inward. In other examples, the locking pins 752A, 752B can be different cross-sectional shapes, such as square or rectangular. The pedicle screw is also surrounded by the pedicle screw receptacle 760 that includes pin openings 762A, 762B to receive the locking pins 752A, 752B, respectively. Pedicle screw receptacle 760 receives the longitudinal bore 770, which extends the length of the counter tensioner 704. The longitudinal bore 770 can allow for insertion of a closure top (e.g., set screw) into the pedicle screw to secure the cord. Finally, FIGS. 7D and 7E illustrate cord guide 710B with opposing cord guide 710A shown in FIG. 7C.

FIGS. 7F and 7G provide cross sectional views of the proximal end 716 of the counter tensioner 704. The cross-sectional views include details of the proximal lock mechanism 780 and interaction with the lock handle 736. The proximal lock mechanism 780 operates to linearly translate the locking extensions 740A, 740B, which in turn shift the pivot locks 750A, 750B from an unlocked to locked position as discussed above. The proximal lock mechanism 780 includes a lock cylinder 782 coupled to the lock handle 736 through two lock cylinder set screws 738A, 738B. The lock handle 736 rotates the lock cylinder 782, which in turn causes translation of the locking extension 740A, 740B. The translation is accomplished, in this example, through interaction between a threaded cylinder 784 and a threaded internal portion of the lock cylinder 782. The threaded cylinder 784 is coupled to the locking extensions 740A, 740B through extension coupling pins 786A, 786B. When the lock handle 736 is rotated, the lock cylinder 782 rotates with an internal threaded surface engaging the external threads on the threaded cylinder 784, which is coupled to the locking extensions. As the lock handle 736 and lock cylinder are coupled to the elongate portion of the counter tensioner 704, the threaded interaction causes the threaded cylinder 784 and locking extensions 740A, 740B to translate in a proximal-distal direction. Other mechanisms for translating the locking extensions 740A, 740B can be utilized without deviating from this basic design, such as a cam follower arrangement between the lock cylinder 782 and locking extensions 740A, 740B.

FIG. 7G also provides illustration of the cord conduit 720 leading to the tensioner port 718. As discussed above, the cord can be routed out of the cord guide 710A, around the cord fulcrum 706, proximally along the cord channel 708, through the cord conduit 720, and into the tensioner 702 through the tensioner port 718.

FIG. 8A is a drawing illustrating a cord tensioning system 800 operating through an auxiliary surgical port 805A, according to various embodiments of the present disclosure. In this example, the cord tensioning system 800 is illustrated as including a plurality of surgical ports 805A, 805B (collectively referenced as surgical port(s) 805), a tensioner 702, a counter tensioner 704, and a tensioner extension 810. The cord tensioning system 800 is illustrated in reference to an exemplary spine 860 with spinal implants 850A-850F (collectively or individually referenced as spinal implant(s) 850). In this example, the spinal implants 850 are pedicle screws with tulip heads adapted to receive a surgical cord (not illustrated). The example illustrated in FIG. 8A is intended to demonstrate, generally, how the cord tensioning system 800 is utilized in reference to a patient’s spine. However, the illustration is not necessarily to scale or anatomically correct, rather it is merely intended to demonstration how the instruments function in combination and relative relationship.

In general, the cord tensioning system 800 is designed to reduce complexity and increase the speed of implanting a surgical cord (e.g., flexible elongate tether) along a deformed portion of a patient’s spine. Prior to creation of the cord tensioning system 800 by the present inventors, implanting a surgical cord involved complex manipulations of the surgical cord through several surgical ports positions directly over the spinal deformity (one or more ports, usually one for every 1 to 3 vertebrae), such as surgical port 805B. The cord manipulations involved securing the cord to a pedicle screw, maneuvering it through the body towards the next pedicle screw, bring the cord up through the surgical port, tensioning the cord, securing the cord to the present pedicle screw, pushing the cord back down through the surgical port and repeating for the next screw. The surgical ports are necessary during this procedure to keep CO2 used to deflate the lung within the body cavity. Manipulating the cord in and out of the surgical ports is a time consuming and challenging part of the surgical procedure.

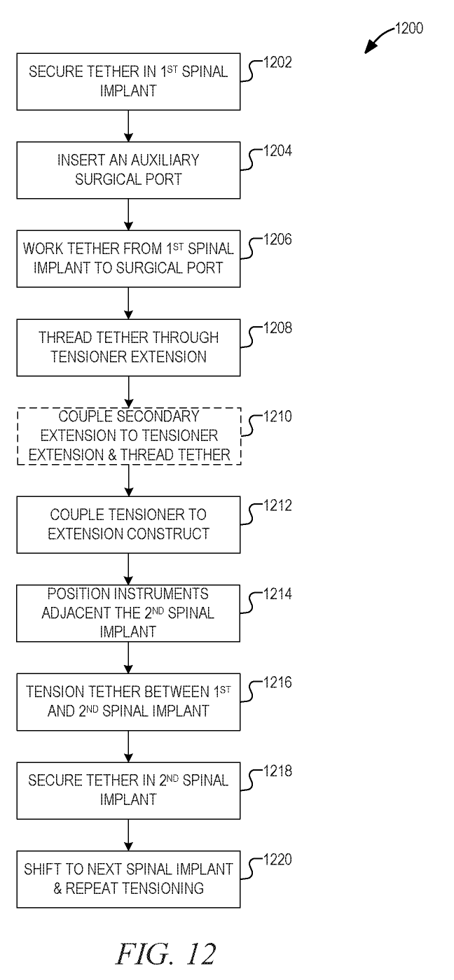

Utilizing the tensioner extension 810 in combination with an auxiliary surgical port, such as surgical port 805A, positioned inferior to the spinal deformity, can avoid maneuvering the surgical cord into and out of a surgical port for each and every spinal implant. In the revised procedure (detailed further in reference to FIG. 12 below), a surgeon threads the surgical cord through the tensioner extension and utilizes the extension to position the surgical cord into each spinal implant and also tension the cord between spinal implants. The revised procedure avoids the need to thread the cord in and out of a surgical port for each and every spinal implant in the deformity correction construct.

In this example, the tensioner extension 810 can include a dual coupler 820 attached to a proximal end of an elongate extension member 830, as well as a nose member 840 attached to a distal end of the elongate extension member 830. As illustrated, one of the functions of the dual coupler 820 is to enable the tensioner 702 to couple to the tensioner extension 810. As discussed below, the dual coupler 820 is also designed to enable connection of a secondary extension to elongate the core tensioning system 800, which allows for the system to operate over a larger deformity construct with minimal additional steps in the surgical procedure. The tensioner extension 810 can be in the range of 300mm to 400mm in length. However, tensioner extensions of different lengths can easily be produced in accordance with this disclosure.

FIG. 8B is a drawing illustrating a cord tensioning instrument 800 including a tensioner extension 810 and secondary extension 870, according to various embodiments of the present disclosure. In this example, the cord tensioning instrument 800 is illustrated with the optional secondary extension 870 coupled between the tensioner 702 and the tensioner extension 810. The secondary extension 870 can provide a surgeon with extra length to reach the most superior spinal implant needed to correct the spinal deformity. The secondary extension 870 is designed to allow for quick connection and disconnection, so that as the surgeon moves closer to the auxiliary surgical port, the overall length of the cord tensioning instrument 800 can be reduced by removing the secondary extension 870 from between the tensioner 702 and tensioner extension 810. Addition or removal of the secondary extension 870 can be performed without re-threading the surgical cord through the tensioner extension 810, which means using it or removing it will not slow down the overall procedure in any significant way.

As illustrated in FIG. 8B, the dual coupler 820 on the tension extension 810 includes structures to enable secure coupling of the secondary extension 870. In this example, the dual coupler 820 can receive the distal extension coupler 890 on the distal end of the short elongate extension member 872 portion of the secondary extension 870. The secondary extension 870 can also include a proximal tensioner coupler 880 affixed to a proximal end of the short elongate extension member 872. In this example, the proximal tensioner coupler 880 includes similar structures as the dual coupler 820 for receiving and securing the nose portion 710 of the tensioner 702. In this example, the short elongate extension member 872 can be a flexible elongate member similar in construction to the elongate extension member 830, but a shorter length. In this example, the secondary extension 870 is in a range of 130mm to 150mm in length. As noted above, the elongate members can be a coil spring or silicone tube material, among other materials.

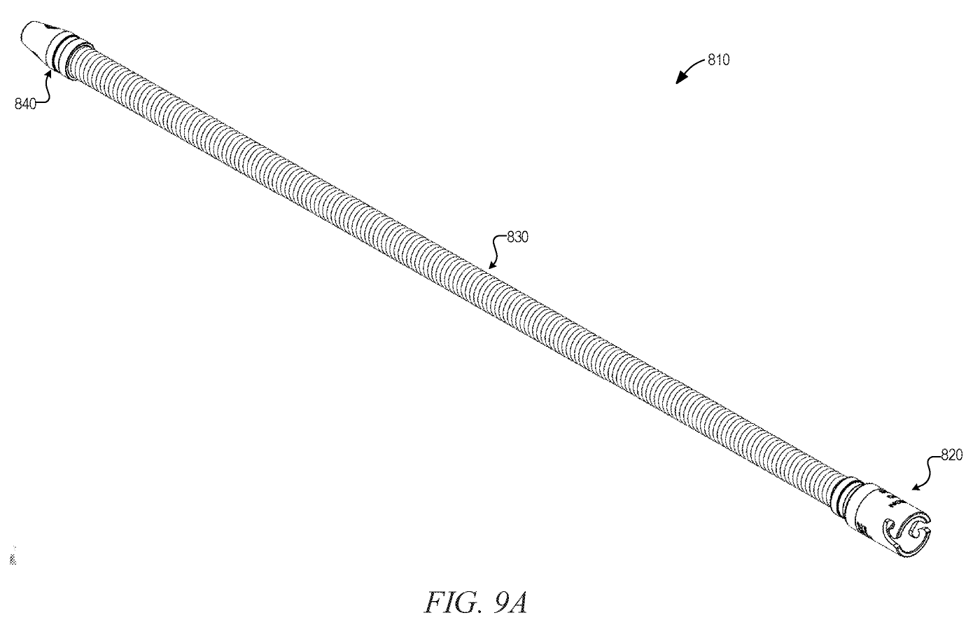

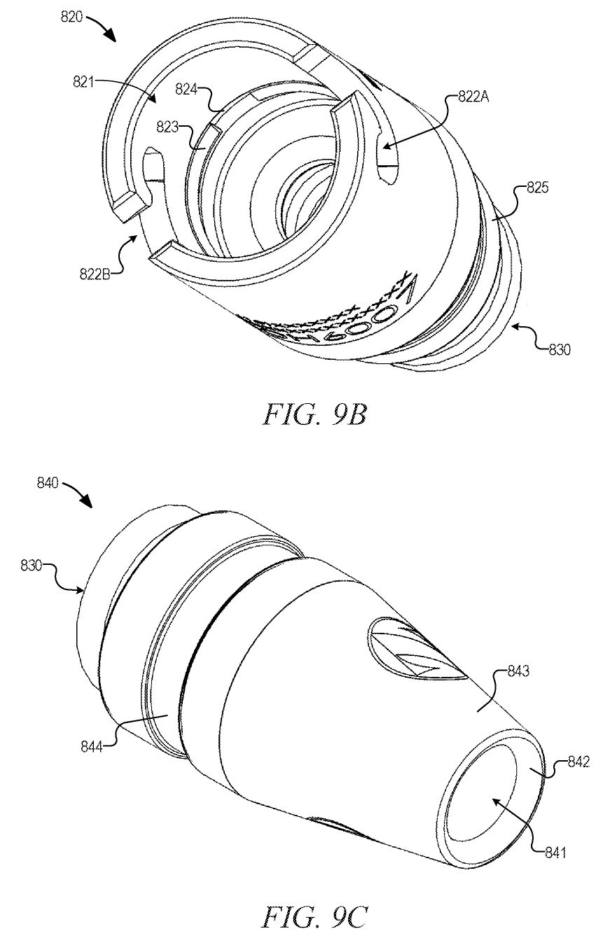

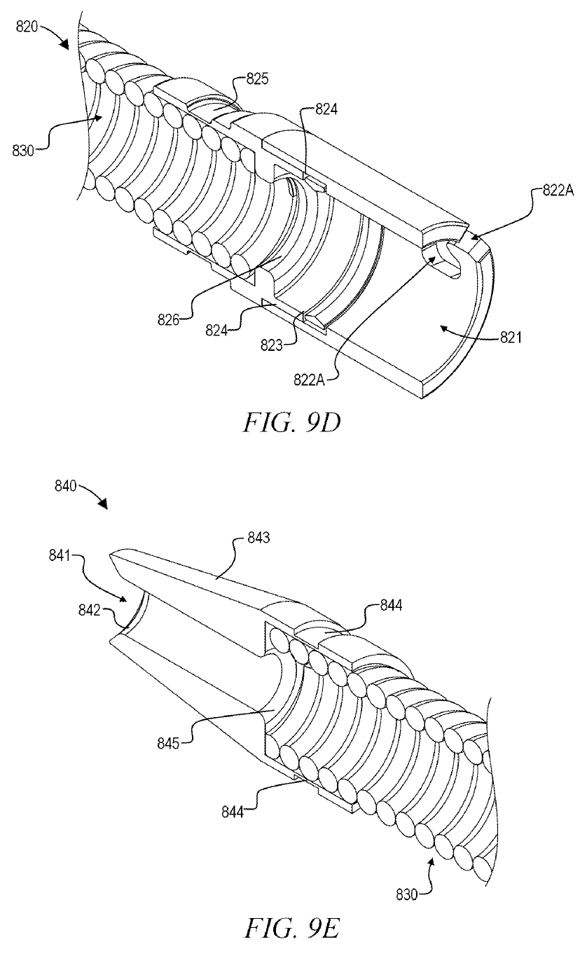

FIGS. 9A-9E are drawings illustrating various aspects of a tensioner extension 810, according to various embodiments of the present disclosure. The following describes the structural components of the tensioner extension 810 in accordance with the example illustrated in FIGS. 9A-9E together. In this example, the tensioner extension 810 includes an elongate extension member 830 between a dual coupler 820 on a proximal end and a distal nose member 840 on the distal end. The illustrated example depicts the elongate extension member 830 as a coil spring with no spacing along the longitudinal length between coils of the coil spring, which allows the construct flexibility and the ability to carry tension along the length of the device. FIG. 9B illustrates a perspective view of the dual coupler 820. In this view, structures such as a coupler bore 821, locking slots 822A, 822B (collectively referenced as locking slot(s) 822), a snap ring 823, a ring groove 824, and a compression member 825. The coupler bore 821 is adapted to receive a nose assembly 710 of the tensioner 702 or a coupler barrel 875 of the secondary extension 870 when the secondary extension 870 is in use. Within the coupler bore 821 is a ring groove 824 with a snap ring 823 disposed within a portion of the ring groove 824. The ring groove 824 is a recess extending around an inner circumference of the coupler bore 821. The snap ring 823 is partially recessed within the ring groove 824, with a rounded or crowned (opposing angles meeting at a raised ridge) surface extending radially into the coupler bore 821. The snap ring 823 only extends partially around the circumference of the ring groove 824, which allows the snap ring 823 to elastically deform. Deformation of the snap ring 823 allows the nose assembly 710 of the tensioner 702 to extend pass the snap ring 823. In some examples, the nose assembly 710 includes a depression or recess extending around the outer circumference that is captured by the snap ring 823 upon insertion into the coupler bore 821.

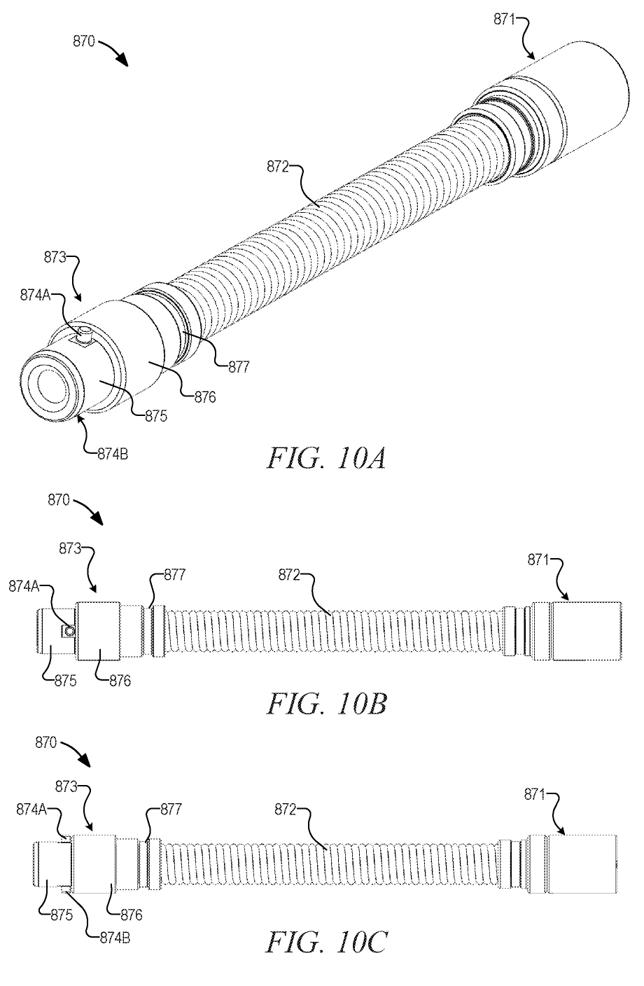

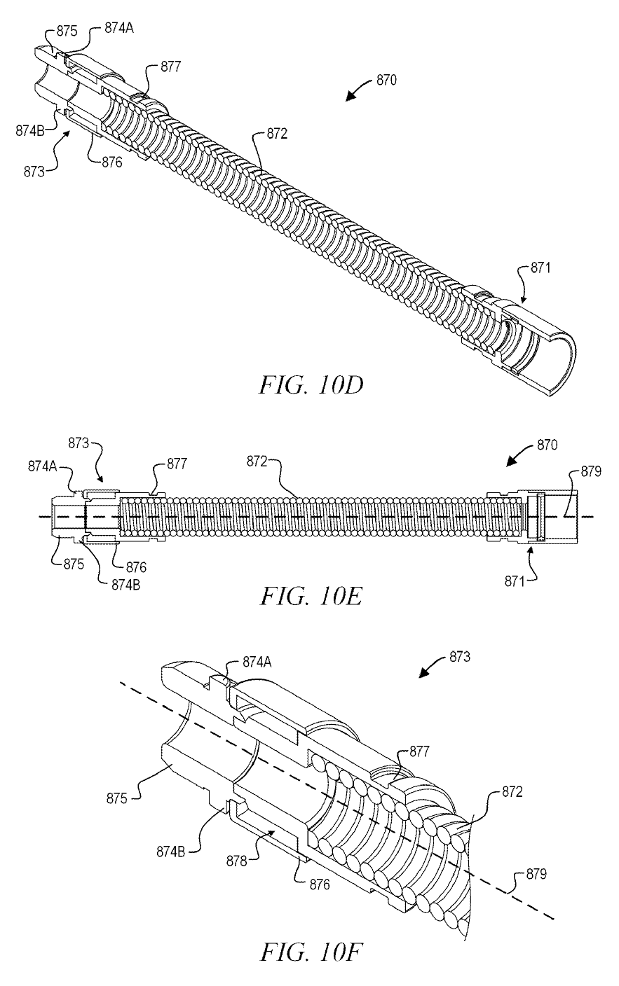

In this example, the dual coupler 820 includes two opposing locking slots 822. The locking slots 822 each include an opening along the outer edge of the dual coupler 820. From the opening, each locking slot includes a P-shaped cut-out extending along a sidewall of the dual coupler 820 (angling around a small portion of the sidewall). The P-shaped cut-out is shaped to receive a locking pin extending from a coupler portion of the secondary extension 870 (illustrated in FIGS. 10A-10F). The locking slots 822 operate in conjunction with the locking pins to enable a twist-lock coupling between the tensioner extension 810 and the secondary extension 870. The twist-lock operation and structure of the locking slots 822 ensures that the secondary extension remains securely attached to the tensioner extension 810 during use, but also enables quick disconnection when desired.

FIG. 9C is a perspective view of an example distal nose member 840. In this example, the distal nose member 840 includes structures such as a nose bore 841, a nose chaffer 842, a tapered body section 843, and a nose compression member 844. The nose bore 841 extends from the distal most end through to a lumen extending the length of the elongate extension member 830. Around the outer edge of the nose bore 841 is a nose chaffer 842, which operates to reduce any friction or wear on the surgical cord exiting the tensioner extension 810. The tapered body section 843 extends proximally away from the distal end ramping up to the transition into the nose compression member 844 that couples the distal nose member 840 to the elongate extension member 830.

FIG. 9D is a cross-sectional view of the dual coupler 820 connected to the elongate extension member 830. The cross-sectional view provides a more detailed view of the snap ring 823 and ring groove 824, as well as a cord guide 826. The cord guide 826 operates to guide the surgical cord smoothly from the tensioner 702 into the elongate extension member 830.

FIG. 9E is a cross-sectional view of the distal nose member 840 connected to the elongate extension member 830. The cross-sectional view details the nose bore 841, the nose chaffer 842, and a nose cord guide 845. The nose cord guide 845 assists in transitioning the surgical cord out of the elongate extension member 830 into the distal nose member 840.

FIGS. 10A-10F are drawings illustrating various aspects of the secondary tensioner extension 870, according to various embodiments of the present disclosure. In this example, the secondary tensioner extension 870 (also referenced simply as the secondary extension 870) includes a secondary extension tensioner coupler 871 coupled to a proximal end of a short elongate extension member 872 as well as an extension coupler 873 capping a distal end of the short elongate extension member 872. The tension coupler 871 includes a snap ring and ring groove comparable to those discussed above in reference to dual coupler 820 for securing the nose assembly 710 of the tensioner 702 (see FIGS. 10D and 10E).

The secondary extension 870 includes extension coupler 873 for securely connecting the secondary extension 871 to the tensioner extension 810. The extension coupler 873 includes locking pins 874A and 874B (collectively referenced as locking pins 874) extending radially away from a coupler barrel 875. As discussed above, the locking pins 874A, 874B interact with locking slots 822 to enable a twist-lock operate to secure the secondary extension 870 to the tensioner extension 810. The locking mechanism can also include a bias cylinder 876, which can be spring loaded or otherwise biased towards the locking pins 874 to assist in securing the secondary extension 870. A spring recces 878 is illustrated in FIG. 10F, but no spring or biasing mechanism is shown within the recess for clarity. The coupler barrel 875 can extend from just proximal of the locking pins 874 to a distal end of the secondary extension 870. The coupler barrel 875 can include a chaffered distal radial edge to ease insertion into the dual coupler 820 as well as to smooth the surgical cord transition on the inner edge.

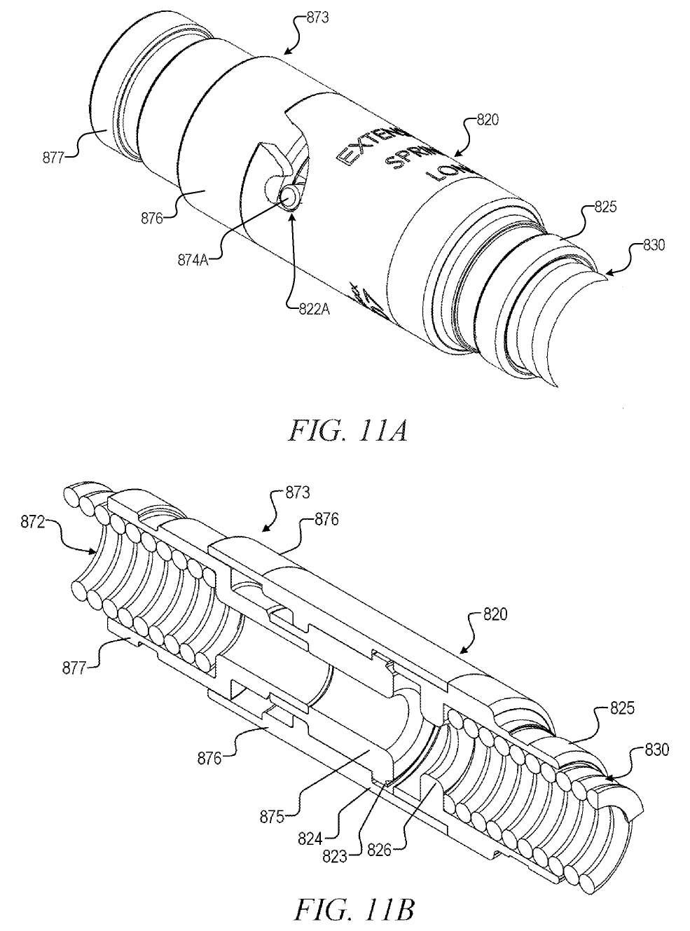

FIGS. 11A-11B are drawings illustrating the connection between the tensioner extension 810 and a secondary tensioner extension 870, according to various embodiments of the present disclosure. In this example, the dual coupler 820 is illustrated as receiving the extension coupler 873, with the locking pins 874 engaged in the P-shaped portion of the locking slots 822. The cross-sectional view illustrates the coupler barrel 875 engaging the snap ring 823 that is disposed in the ring groove 824. In certain examples, both the coupler barrel 875 and the nose assembly 710 include a detent running around the outer circumference and adapted to engage the snap ring 823 upon insertion.

FIG. 12 is a flowchart illustrating surgical procedure 1200 for tensioning a surgical cord utilizing tensioner 702, tensioner extension 810, and optionally a secondary extension 870, according to various embodiments of the present disclosure. In this example, the surgical procedure 1200 can include operations such as: securing a cord in a 1st spinal implant at 1202, inserting an auxiliary surgical port at 1204, working the cord from the 1st spinal implant to the auxiliary surgical port at 1206, threading the cord through a tensioner extension at 1208, optionally coupling a secondary extension and threading the cord at 1210, coupling a tensioner to the cord tensioning instrument at 1212, positioning instrument adjacent a 2nd spinal implant at 1214, tensioning the cord at 1216, securing the cord in the 2nd spinal implant at 1218, and repeating the positioning and tensioning process for all remaining spinal implants at 1220. In the following discussion, the core tensioning instrument discussed can include the tensioner 702, the tensioner extension 810, and optionally the secondary extension 870. Further, throughout the disclosure the terms tether and cord are used to refer to a flexible, optionally elastic, surgical cord extendable between spinal implants, such as pedicle screws.

In this example, the surgical procedure 1200 can begin at 1202 with a surgeon working through a first surgical port, such as surgical port 805B, to secure a first end of a surgical cord to a 1st spinal implant. The 1st spinal implant in this example is the most superior spinal implant being used to correct the spinal deformity. Other examples may include working in an opposite direction and include use of an auxiliary surgical port that enters the patient superior to the spinal deformity. However, in the present example, an auxiliary surgical port, such as surgical port 805A, enters the patient inferior to the spinal deformity, such as in the area of the navel or a lateral position in the abdomen. The surgical procedure 1200 can continue at 1204 with insertion of an auxiliary surgical port, such as surgical port 805A. Additionally, while the surgical procedure 1200 is discussed utilizing surgical ports that operate to provide access and seal the body cavity, the surgical procedure could potentially be performed using more traditional surgical retractors. Accordingly, the type of surgical port or retractor is not material to the described operations involving the cord tensioning instrument 800. However, where surgical ports as described are necessary, such as to retain CO2 within the body cavity, the cord tensioning instrument 800 further enhances a surgeon’s ability to perform the required steps.

At 1206, the surgical procedure 1200 can continue with the surgeon working/maneuvering the cord from the 1st spinal implant up through the auxiliary surgical port. At 1208, the surgical procedure 1200 can continue with the cord being threaded through a cord tensioning instrument, such as cord tensioning instrument 800 (previously referenced as cord tensioning system 800). At this stage of surgical procedure 1200, the cord tensioning instrument 800 can include at least the tensioner extension 810. At 1210, the surgical procedure 1200 can optionally include coupling a secondary extension, such as secondary extension 870, to the cord tensioning instrument 800. In an example, coupling the secondary extension 870 involves threading the secondary extension over the cord, inserting the coupling barrel 875 into the dual coupler 820, and locking the locking pins 874 into the locking slots 822. Once, the desired extension length is configured for the core tensioning instrument 800, the surgical procedure 1200 can continue at 1212 by coupling tensioner 702 onto the cord tensioning instrument 800. Note, in some examples, the surgeon may delay coupling the tensioner 702 to the core tensioning instrument 800 if maneuvering the cord within the patient is easier without the tensioner 702 attached. The tensioner 702 can be attached and detached as needed to facilitate the procedure.

At 1214, the surgical procedure 1200 can continue with the surgeon positioning instruments, such as a distal end of the cord tensioning instrument 800 and a counter tensioner 704 adjacent a 2nd spinal implant. In this step, the distal nose member 840 of the tensioner extension 810 can be used to insert the cord into the 2nd spinal implant and then be positioned adjacent to the 2nd spinal implant for tensioning the cord. The counter tensioning 704 can assist in positioning the cord and is also used to insert a set screw later in the procedure.

At 1216, the surgical procedure 1200 can continue with the surgeon using the cord tensioning instrument 800 to tension the cord between the 1st and 2nd spinal implant. Tensioning occurs through the surgeon articulating the tensioner 702, which pulls on the cord, while counter tension is carried through the tensioner extension 810 to the side of the 2nd spinal implant (see FIG. 8A). Once the desired amount of tension has been applied, the surgical procedure 1200 can continue at 1218 with the surgeon inserting a set screw into the 2nd spinal implant to secure the cord. In this example, the counter tensioner 704 can be utilized to deliver the set screw through a surgical port into the spinal implant.

Finally, the surgical procedure 1200 can continue at 1220 with the surgical team shifting the instruments to the next spinal implant, such as a 3rd spinal implant, and repeating operations 1214 to 1218. One of the benefits of utilizing the cord tensioning instrument 800 involves not having to repeatedly maneuver the cord in and out of a surgical port for each spinal implant. Rather, with surgical procedure 1200 the cord is only maneuvered up through the auxiliary surgical port once. In some examples, tensioning and securing the cord to the final spinal implant, such as spinal implant 850F, may be performed using the counter tensioner 704 in combination with tensioner 702 with the cord threaded up through a surgical port positioned above the spinal implant.

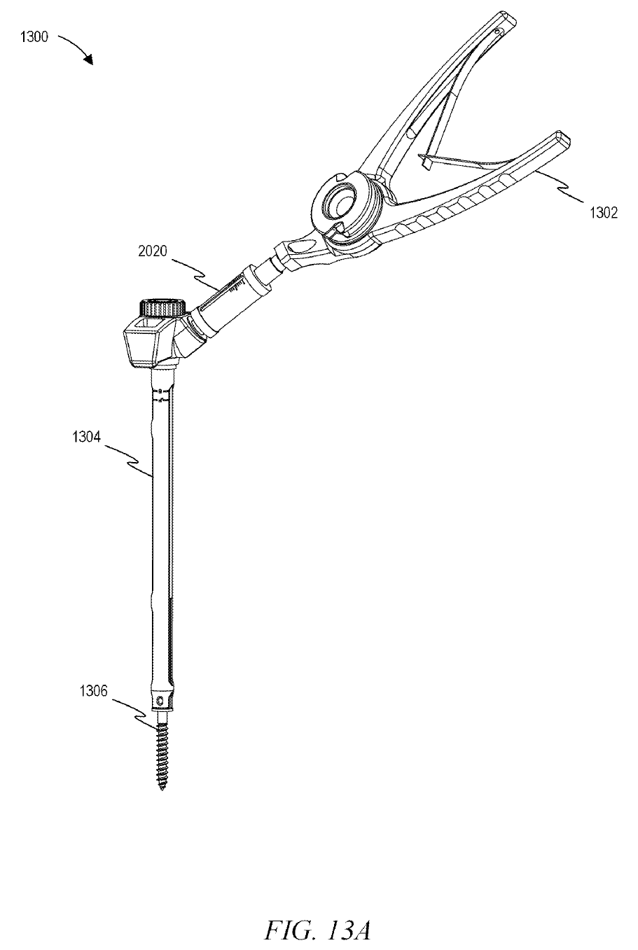

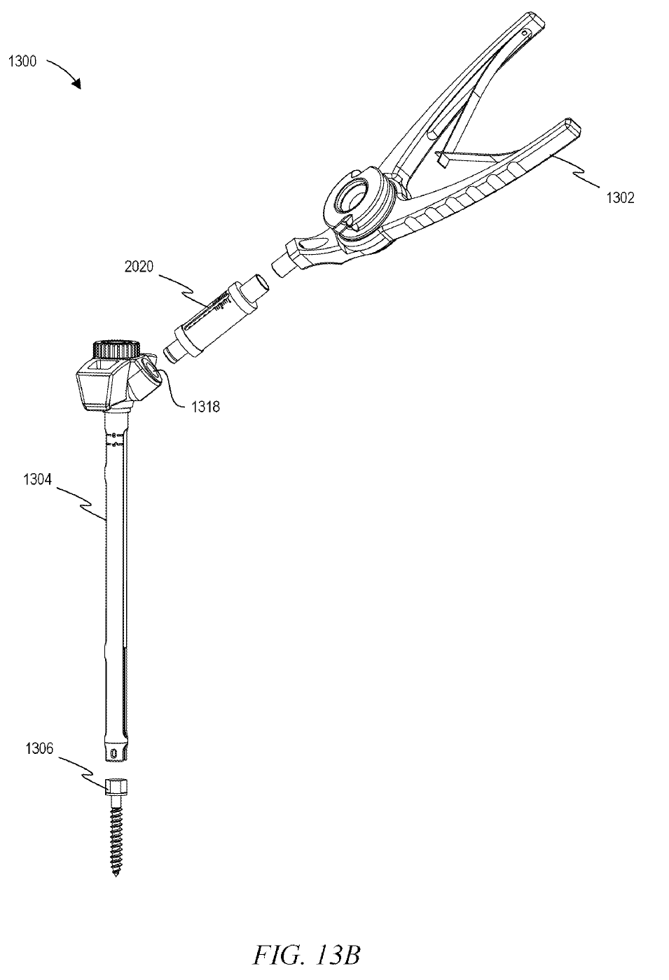

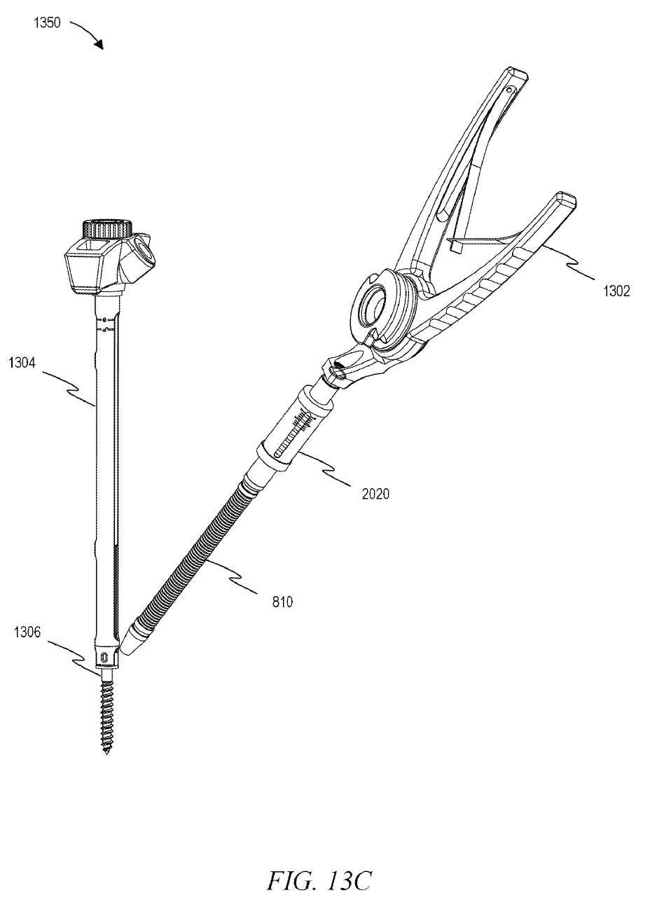

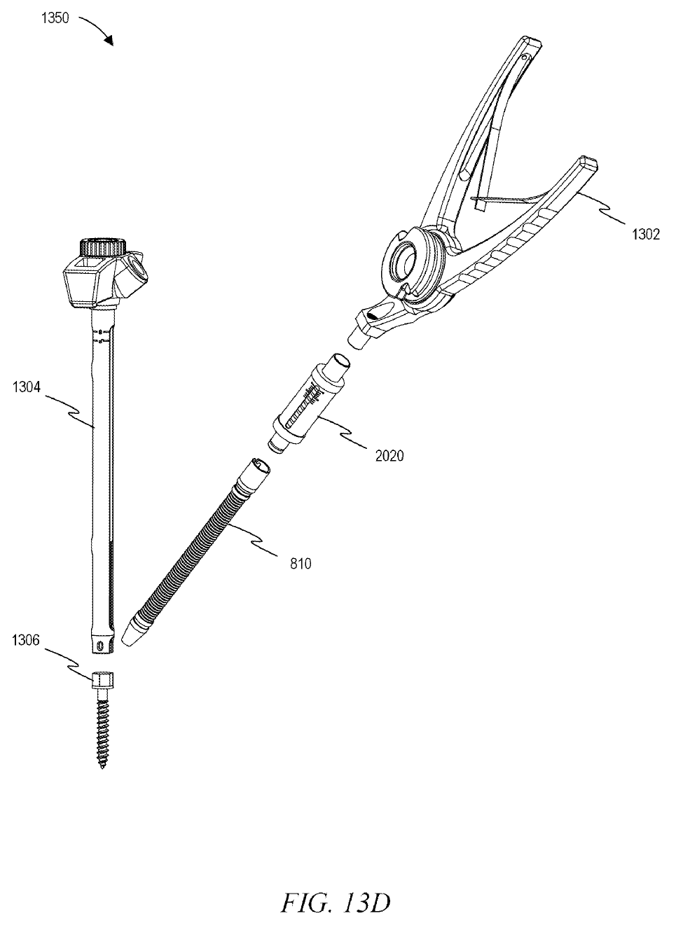

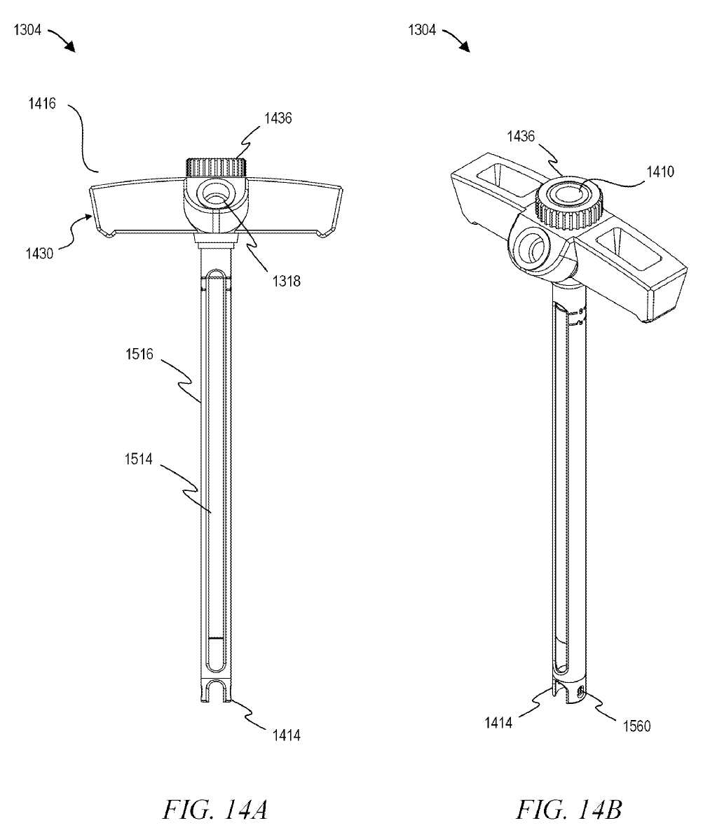

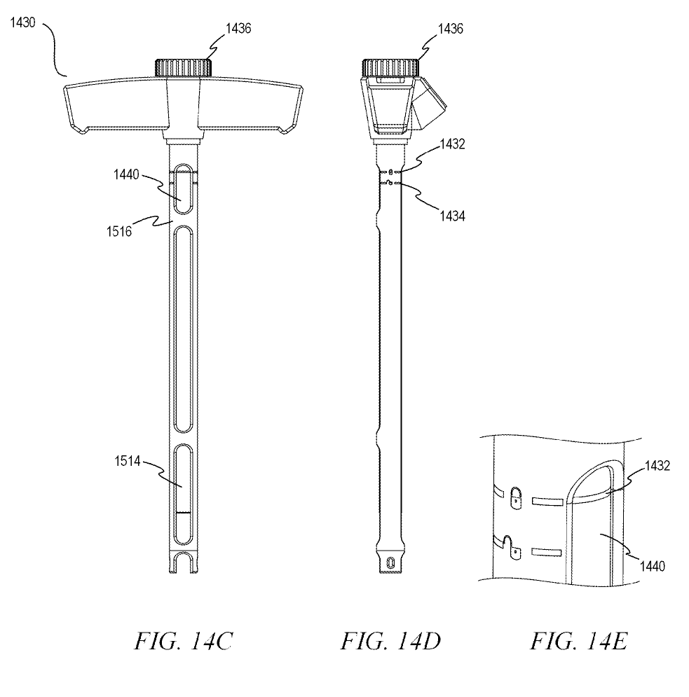

Turning now to FIGS. 13A and 13B, an exemplary embodiment of a system 1300 according to the present disclosure is illustrated. System 1300 can comprise a wheel (reel) tensioner 1302, a tension indicator 2020, and a counter tensioner 1304, which can be used in combination for manipulating implants 1306 couplable by a cord (tether) 108 (not shown). The wheel tensioner 1302 is designed to enable one-handed operation with the hand in an ergonomic position to apply tension to a cord 108. The tensioner 1302 can comprise a nose 1310 for receiving the cord 108 and a cord wheel 1312 for securing and applying tension to the cord 108. The counter tensioner 1304 can be releasably couplable to a head of an implant 1306 at a distal end 1414 (FIGS. 14A-14B) thereof and can have a port 1318 disposed at a proximal end 1416 (FIGS. 14A-14B) thereof. The port 1318 can be releasably couplable to the nose 1310 of the tensioner 1302. The counter tensioner 1304 can further be rigidly couplable to both the head of the implant 1306 and the nose 1310 of the wheel tensioner 1302, allowing a surgeon to tension the cord 108 via the wheel tensioner 1302 and translate one or more implants 1306 and underlying vertebrae relative to one or more other implants implanted in adjacent or nearby vertebrae via a handle 1430 in the same surgical step and as explained in greater detail below.

FIGS. 13C and 13D illustrates a cord tensioning system 1350 operating through an auxiliary surgical port 805A (shown in FIG. 8A), according to various embodiments of the present disclosure. In this example, the cord tensioning system 1350 is illustrated as including a plurality of surgical ports 805A, 805B (see FIG. 8A, collectively referenced as surgical port(s) 805), a tensioner 1302, a counter tensioner 1304, and a tensioner extension 810. In this example, one or more spinal implant 1306 are pedicle screws with tulip heads adapted to receive a surgical cord (not illustrated). The example illustrated in FIGS. 13C and 13D is intended to demonstrate, generally, how the cord tensioning system 1350 is utilized in reference to a patient’s spine as shown in FIG. 8A. However, these illustrations are not necessarily to scale or anatomically correct, rather it is merely intended to demonstrate how the instruments function in combination and relative relationship.

In general, the cord tensioning system 1350 is designed to reduce complexity, improve the ergonomics of the surgical instrumentation and increase the speed of implanting a surgical cord (e.g., flexible elongate tether) along a deformed portion of a patient’s spine. Prior to creation of the cord tensioning system1350 by the present inventors, implanting a surgical cord involved complex manipulations of the surgical cord through several surgical ports positioned directly over the spinal deformity (one or more ports, usually one for every 1 to 3 vertebrae), such as surgical port 805B as shown in FIG. 8A. The cord manipulations involved securing the cord to a pedicle screw, maneuvering it through the body towards the next pedicle screw, bringing the cord up through the surgical port, tensioning the cord, securing the cord to the present pedicle screw, pushing the cord back down through the surgical port and repeating for the next screw. The surgical ports are necessary during this procedure to keep CO2 used to deflate the lung within the body cavity. Manipulating the cord in and out of the surgical ports is a time consuming and challenging part of the surgical procedure. Such a method entailed not just a longer surgery, but also recovery period due to the length of the surgery, but also the stress on the patient’s body of the extensive manipulations of the cord involved in the method.

Utilizing the tensioner extension 810 in combination with an auxiliary surgical port, such as surgical port 805A, positioned inferior to the spinal deformity, can avoid maneuvering the surgical cord into and out of a surgical port for each and every spinal implant. In the revised procedure (discussed generally in reference to FIG. 12), a surgeon threads the surgical cord through the tensioner extension and utilizes the extension to position the surgical cord into each spinal implant and also tension the cord between spinal implants. The revised procedure avoids the need to thread the cord in and out of a surgical port for each and every spinal implant in the deformity correction construct. This procedure potentially improves surgical and patient recovery time and is discussed in more detail with regards to FIGS. 8A-12.

In the present example as shown in FIGS. 13C and 13D, the tensioner extension 810 can be releasably coupled to tension indicator 2020, which is coupled to wheel tensioner 1302. In this example, the tensioner extension 810 is positioned at each spinal implant 1308, as counter tensioner 1304 is coupled to the spinal implant 1306 to provide a counter force as the wheel tensioner 1302 is tensioned until the predetermined tension is indicated by the tension indicator 2020, at which time a set screw (not illustrated) will be delivered via the central lumen of the counter tensioner 1304, securing the cord at that spinal implant 1306. The surgery will then continue to the next vertebrae in the surgical construct until all relevant levels are tensioned appropriately and the cord is secured.

Counter tensioner 1304 is illustrated in various perspective views in FIGS. 14A through 14D. Counter tensioner 1304 can include a handle 1430 at the proximal end 1416 thereof; an elongate member that has an outer shaft 1516 and an inner shaft 1514; and an assembly configured to releasably couple to the head of a spinal implant 1306. The distal end 1416 or the handle 1430 can include a port 1318 that is configured at an angle from the elongate shaft 1516. The counter tensioner port is configured to releasably receive the tension indicator 2020 or the tensioner 1302 if the system is being utilized without the tensioner extension members 810 and 870. While the system 1300 is configured for the use of a tension indicator coupled between the tensioner 1302 and the counter tensioner 1304, it is contemplated that some surgeons may develop a tactile feel of the tension created by utilizing the wheel tensioner to determine the predetermined tension, or there may be levels in a construct that require minimal tensioning and according a surgeon may elect to forgo the tension indicator 2020 at one or more levels of a construct, in which case, the counter tensioner port 1318 is also configured to receive the nose 1602 of tensioner 1302 directly, as well as the tension indicator 2020.

Counter tensioner 1304 can include a central lumen 1410 that runs from the proximal end 1416 to the distal end 1414. The central lumen 1410 of the counter tensioner 1304 can be configured to receive a locking member or set screw and a driver, such that when the cord 108 is tensioned by the tensioner 1302 – either via the counter tensioner port 1318 or via the tensioner extension system 800 - in the head of the spinal implant 1306, a set screw attached to a driver (not shown) can be inserted into the central lumen and the cord 108 can be secured to the head of the spinal implant 1306 during surgery. The counter tensioner 1304 can include engagement members at the distal end 1414 of the counter tensioner 1304 releasably engage with cooperative features on the head of the spinal implant 1306 when the lock knob 1436 at the proximal end 1416 is turned, causing the inner shaft 1514 to slide upwards within the outer shaft 1516, which urges engagement members at the distal end 1414 of the inner shaft 1514 to engage with the cooperating features on the head of the spinal implant 1306.

The counter tensioner 1304 may further include a lock indication 1432 and an unlocked indication 1434 on the outer shaft 1516 and a corresponding locked/unlocked indication 1436 on the inner shaft 1514. When the counter tensioner 1304 is in the unlocked position, the inner shaft 1514 is in a lower position within the outer shaft 1516 and the locked/unlocked indication 1436 on the inner shaft 1514 visible and is aligned with the unlocked indication 1434 on the outer shaft 1516 via a window 1440 near the proximal end 1318 of the counter tensioner 1304. In operation, when the lock knob 1436 is turned, causing the inner shaft 1514 to slide upwards within the outer shaft 1516 and the engagement members at the distal end 1414 to become engaged, as the inner slides upward within the outer shaft 1516, the locked/unlocked indication 1436 moves upward to visibly align next to the locked indication 1432 via the window 1440 in the outer shaft 1516. It will be readily appreciated by one of ordinary skill that the outer shaft 1516 could slide, rather than the inner shaft 1514 sliding, that the locked/unlocked indicators could be reversed between the inner shaft 1514 and outer shaft 1516, or that other methods of visibly indicating the locked versus unlocked status of the engagement members could be altered without departing from the concepts described herein.

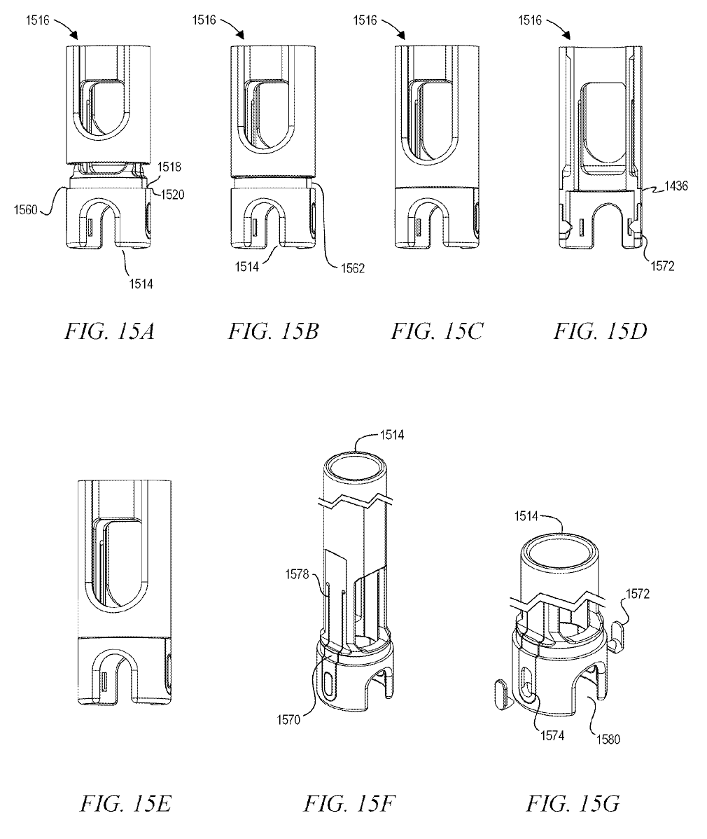

Other exemplary aspects of the counter tensioner 1304 are illustrated in FIGS. 15A-15G which show various close-up views of the distal end 1414 of the counter tensioner1304 where a number of features discussed above are shown in further detail. Inner shaft 1514 can include a leaf spring or living hinge 1570 at the distal end. The living hinge 1570 can include mushroom pins 1572. The living hinges 1570 may be made via laser cutting, wire cutting or other known methods of manufacturing living hinges. The mushroom pins 1572 may be laser welded, glued or otherwise attached to the living hinges 1570. This aspect would permit the locking elements of the counter tensioner 1304 to be a solid body with fewer components. The mushroom pins are shaped and sized such that the inner surface of the mushroom pins 1570 engage cooperating engagement features on the head of the spinal implant 1306 when the inner shaft 1514 is slid upwards within the outer shaft1516 of the counter tensioner 1304, causing the outer shaft to hold the living hinges 1570 and thus the mushroom pins 1572 in a locked position on the head of the spinal implant 1306. In the locked position, the counter tensioner 1304 is rigidly matted to the head of the spinal implant 1306 and the counter tensioner is capable of being utilized as part of the tensioning of the cord 108. When the counter tensioner 1304 is in the unlocked position, the living hinges 1570 are able to flex and move via the laser cut channels 1578, permitting the mushroom pins 1572 to flex into and out of engagement with the cooperating or corresponding engagement features (not shown) on the spinal implant 1306.

The counter tensioner 1304 may further include a ledge 1560 on the inner shaft 1514 at the distal end 1414 defined between a first diameter 1518 and a second diameter 1520. When the lock knob 1436 is turned, causing the inner shaft 1514 is slide upwards within the outer shaft 1516, the movement is stopped when the outer shafter 1516 comes into contact with the ledge 1560 of the inner shaft. In this locked position, the distal end of the outer shaft 1516 rests on the ledge 1560 of the inner shaft 1514 and the living hinges 1570 are prevented from flexing or moving outwards. Thus, in the locked position, the mushroom pins will engage the corresponding engagement features on the spinal implant 1306 and the counter tensioner 1304 will be rigidly attached to the head of the spinal implant 1306 until the lock knob is turned in the unlocking direction causing the inner shaft to slide downwards, moving the distal end of the outer shaft 1516 from the locked position on the ledge 1560 of the inner shaft 1514 and the living hinges 1570 will be free to flex outwards so that the mushroom pins 1572 are able to flex out of the way of the corresponding engagement features on the spinal implant 1306 and the counter tensioner can be removed from the spinal implant 1306. Beyond the locked/unlocked indications at the proximal end 1416 of the counter tensioner 1304, inner shaft 1514 may further include a visual indication at the distal end 1414 of the inner shaft 1514, such as a different color 1562 immediately above the ledge 1560 to visually indicate that the counter tensioner 1304 is in the unlocked or locked position.

The counter tensioner 1304 may further include a locking limitation, such as an inner diameter 1580 of a portion of the distal end of the outer shaft 1516 having a close fit with the inner shaft wall adjacent to the ledge 1560. For example, the inner diameter 1580 of the outer shaft 1516 closely fits over the first diameter 1520 defining a portion of the ledge 1560. Consistent with this embodiment, if the mushroom pins 1572 are over the head of the spinal implant 1306 but are not engaged in the corresponding engagement features of the implant 1306, the mushroom pins 1572 will be flexed outward on the living hinges 1570 causing the living hinges 1570 to extend beyond the first diameter 1520, and the inner shaft 1514 will be prevented from moving into the locked position due to the inner diameter 1518 of the outer shaft 1516 not being able to slide over the first diameter 1520 of the inner shaft 1514. Accordingly, the counter tensioner 1304 will be unable to be moved into the locked position if the mushroom pins 1572 are correctly engaged or seated in the corresponding engagement features on the spinal implant 1306.

The counter tensioner 1304 may further include channels 1580 for receiving the cord 108, which enable the counter tensioner to assist with aligning the cord 108 into the tulip head of the spinal implant, so that a driver with a set screw can be inserted into the central lumen 1410 of the counter tensioner 1304 and secure the cord 108 into the tulip head of the spinal implant 1306.

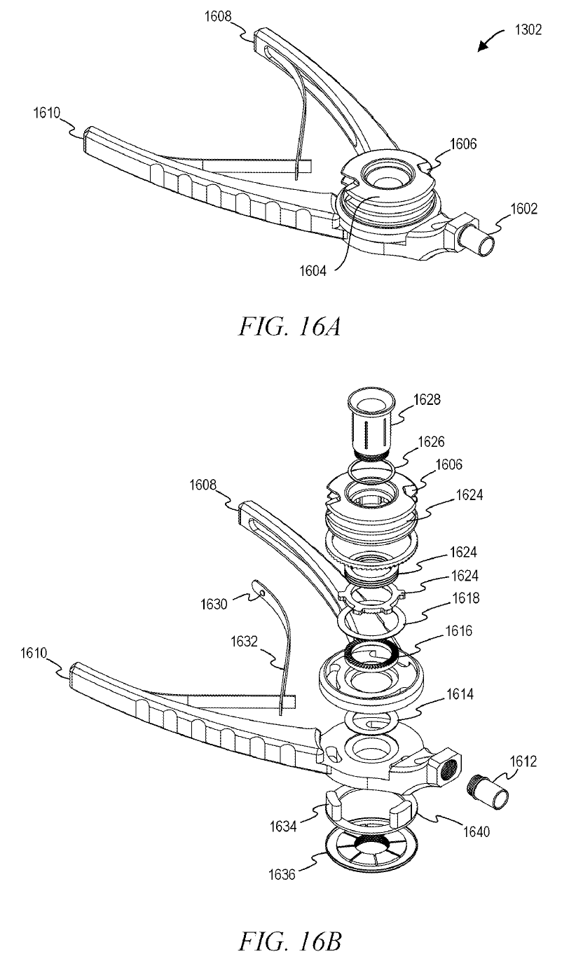

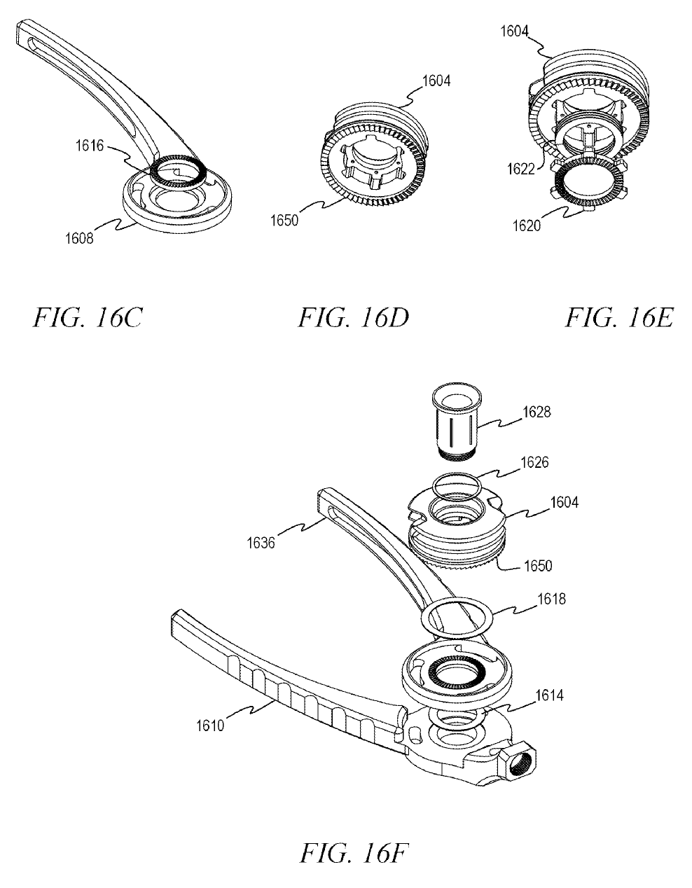

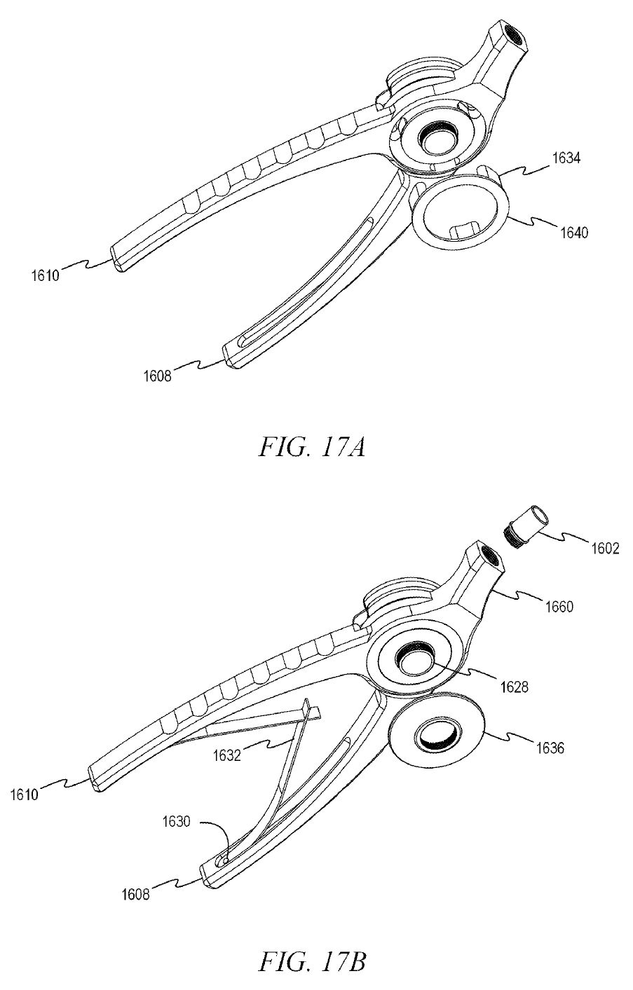

An exemplary embodiment of the tensioner 1302 is discussed with respect to FIGS. 16A-16F illustrating tensioner 1302 that can have a nose 1602 with a lumen 1612 for receiving a cord (tether), a cord wheel (cord reel) 1604 with one or more cord retention notches 1606, a first handle 1608 and a second handle 1610. The assembly of cord tensioner 1302 will be discussed with respect to FIGS. 16A through 17B.

The assembly can begin by pressing a free hub short 1616 in the first handle 1608 central opening, aligning the parts so the key features line up, creating a first handle sub assembly (FIG. 16C). The assembly can continue by aligning key features and pressing cord wheel teeth 1650 into the cord wheel 1604 (FIG. 16D). The cord tensioner assembly may continue by aligning and inserting a free hub spring 1622 and a cord wheel free hub 1620 into the cord wheel 1604, creating a cord wheel sub assembly (FIG. 16E). The cord tensioner assembly can be further continued by aligning and inserting a pressure ring 1614 into a corresponding cut out in the second handle 1610; aligning center holes and placing the first handle 1608 directly on the pressure ring 1614; aligning and placing a hub pressure ring 1618 on top of the sub assembly of the first handle 1608 and free hub short 1616 from the an earlier step (FIG. 16C); aligning the cord wheel sub assembly (FIG. 16E) onto a hub pressure ring 1618; aligning and inserting a center post 1628 with a center pressure ring 1626 through a center hole of all of the prior assembled components (FIG. 16F).

The assembly of the cord wheel tensioner 1302 may be continued on the underside of the tensioner by aligning and inserting slot pawls 1634 attached to a pawl ring 1640 into corresponding slots in the second handle 1610 followed by the pawl spring 1638 (FIG. 17A). The assembly may continue by threading a cap 1636 onto the center post 1628 and securing with any known locking adhesive, such as thread locker and capturing the sub assembly of FIG. 17A. Next in the assembly process, the tensioner nose 1602 may be threaded into the front of the second handle 1610, securing with any known locking adhesive, such as thread locker. The tensioner assembly may continue by securing handle leaf springs 1632 into corresponding slots on both the first and second handles 1608, 1610, respectively, securing in place with leaf spring screws 1630 (FIG. 17B).

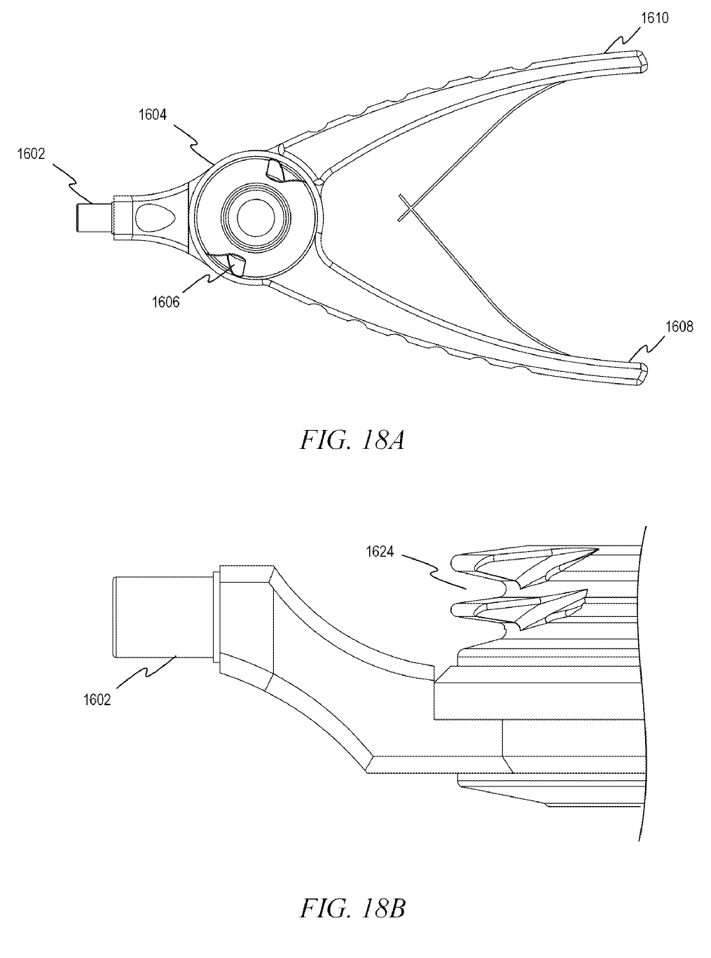

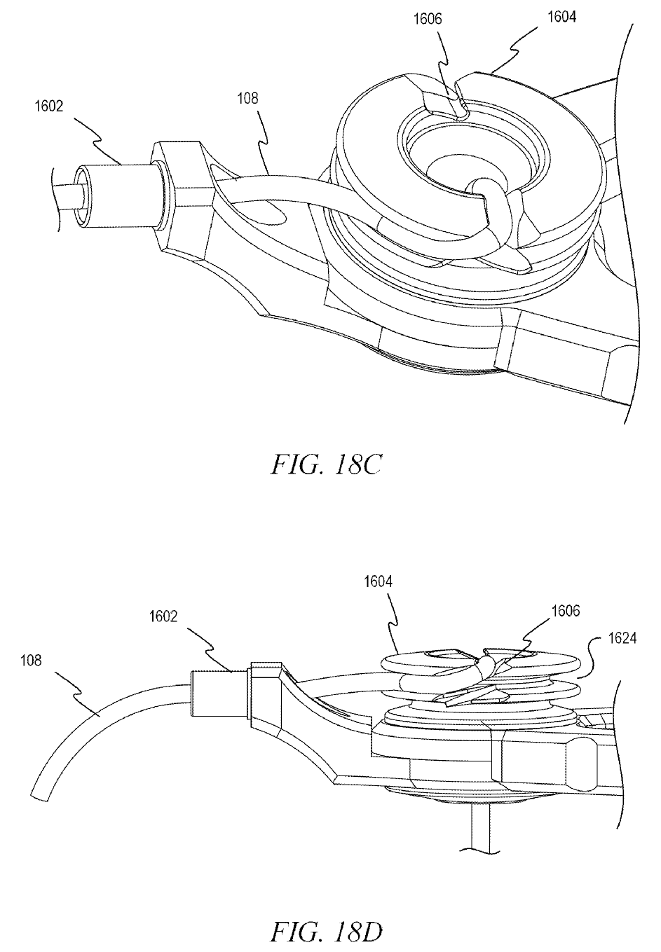

FIGS. 18A-18B illustrate exemplary aspects of the cord wheel tensioner 1302 with first handle 1608, second handle 1610, the nose 1602 for receiving a cord 108, and the cord wheel (reel) 1604 with cord retention notches 1606 and cord alignment grooves 1624. FIG. 18A also illustrates the cord wheel 1604 with arrows indicating the direction the cord wheel turns when being tensioned by squeezing the first and second handles together.

The cord wheel tensioner 1302 may receive a cord (tether) through the lumen in nose 1602 and winding the cord 108 around the cord alignment grooves 1624 of the cord wheel 1604, threading the cord 108 through one of the cord retention notches 1606, and threading the cord 108 through the center post 1628 hole of the cord wheel 1604 as shown in FIGS. 18C-18D.

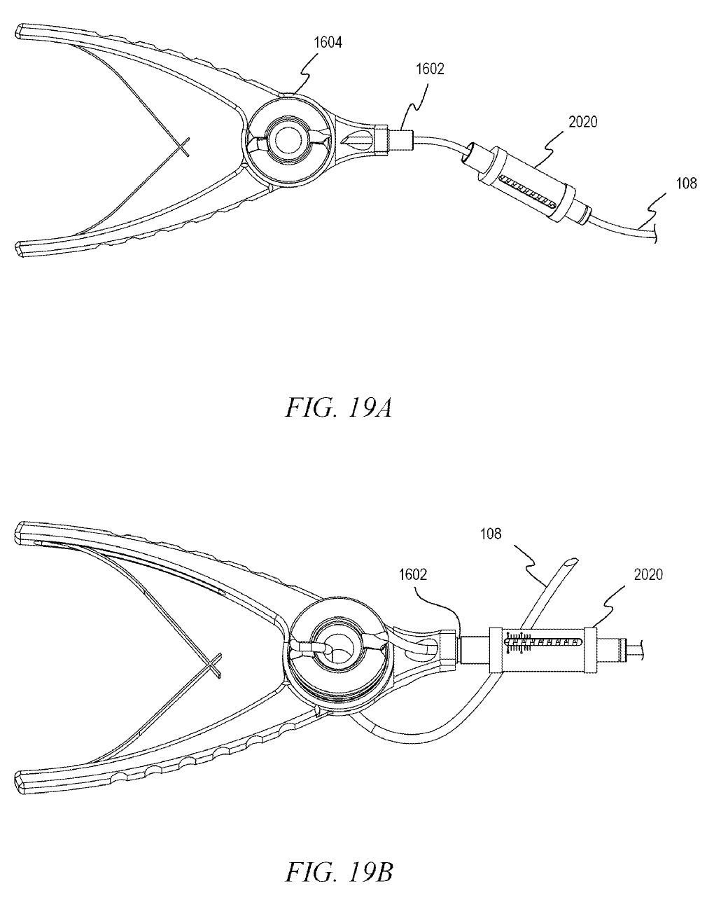

In another exemplary aspect, the cord wheel tensioner 1302 may be utilized in combination with the cord tension indicator 2020 as shown in FIGS. 19A-19B. In this example, cord 108 is threaded into the tension indicator 2020 and into the cord receiving lumen 1612 of nose 1602 of the wheel tensioner 1302. The cord 108 is then wound around the cord alignment grooves 1624 and into a cord retention notch 1606 and through the center post hole 1628 of the cord wheel 1604 (FIGS. 19A-19B).

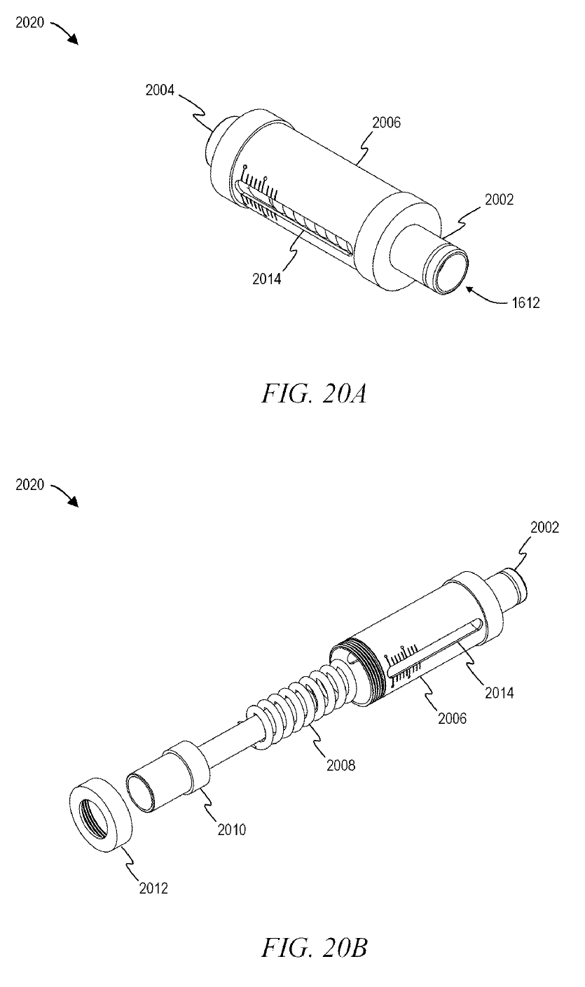

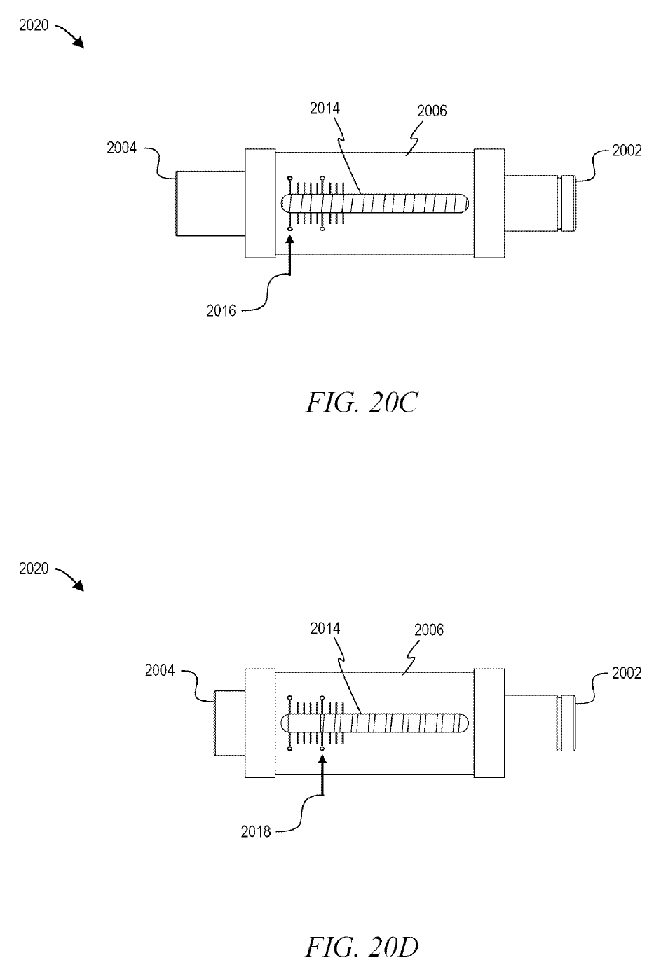

As shown in FIGS. 20A-20D, the cord tension indicator 2020 may comprise a main housing 2006 configured to accommodate a tension spring 2008 and a spring inner shaft 2010, which may be secured in the main housing 2006 with an end cap 2012. The cord tensioner indicator 2020 may also comprise a first port 2002 configured to be received by and mate with the counter tensioner port 1318 or the tensioner extension 810 port and a second port 2004 configured to receive and mate with the nose 1602 of the cord wheel tensioner 1302. The cord tension indicator may further comprise a central lumen 1612 therethrough for receiving cord 108. The cord tension indicator 2020 may also include a tension indicator display window 2014 through which the tension spring 2008 is visible. The tension indicator window 2014 may also show various tension indicator markings, which may include a zero-tension indication 2016 and a maximum tension indication 2018.

The cord tension indicator may be assembled by first inserting the tension spring 2008 and the spring inner shaft 2010 into the spring housing 2006. The end cap 2012 may then be threaded on the spring housing 2006 and secured with any known thread adhesive, such as thread locker to securely capture the components of the tension indicator 2020 (FIG. 20B).

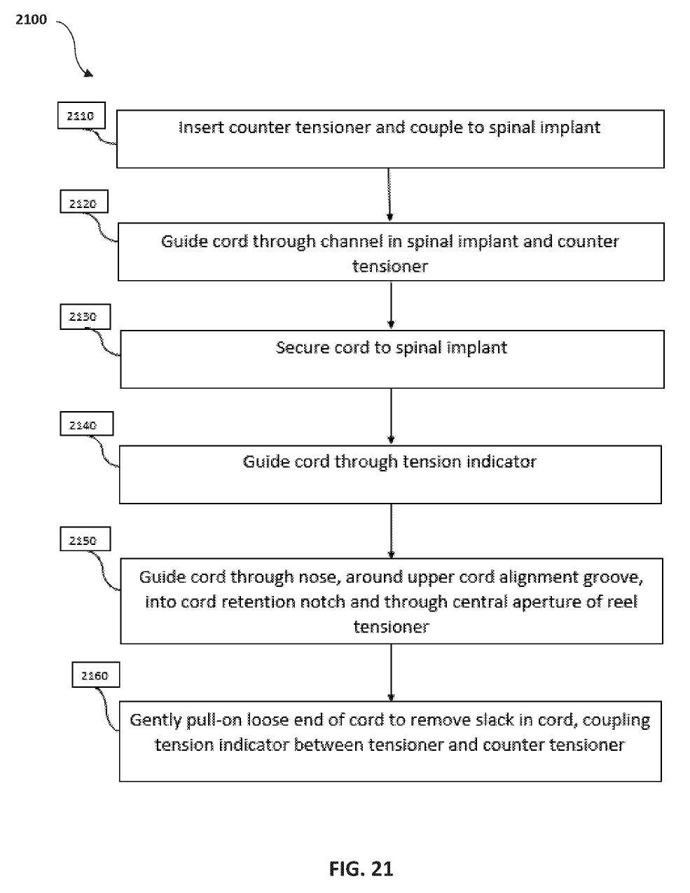

FIG. 21 is a flowchart illustrating an exemplary method 2100 for loading a cord and coupling the counter tensioner, tension indicator and tensioner according to an aspect of the disclosure. The method 2100 may include operations such as inserting and coupling a counter tensioner to a spinal implant 2110; guiding a cord (tether) through a channel in the head of a spinal implant and counter tensioner 2120; securing a cord to the spinal implant 2130; guiding the cord through a tension indicator 2140; guiding the cord through a nose of a tensioner, winding the cord around cord alignment grooves, into a cord retention notch, and through a central aperture of a wheel tensioner 2150; and gently pulling on the loose end of the cord to remove any slack in the cord and causing the counter tensioner, tension indicator and tensioner to be coupled 2160.

The method 21 may begin at 2110 with a counter tensioner, such as counter tensioner 1304 being inserted through an incision and coupled to a head of an implant 1306. In an example, the counter tensioner 1304 includes an inner shaft 1514 having living hinges 1570 with mushroom pins1572 that engage with corresponding engagement features on the head of spinal implant 1306 when a lock knob 1436 at the proximal end of the counter tensioner 1304 is turned, causing the outer sleeve to slide down over the inner sleeve, locking the living hinges in place and mushroom pins 1572 in rigid engagement with corresponding engagement features on the head of the spinal implant 1306. In this example, at 2120, cord (tether) 108 can be guided through cord channel 1580 at the distal end 1404 of counter tensioner 1304 and corresponding channel in the head of the spinal implant 1306. At 2130, the cord 108 may then be secured to the head of the spinal implant 1306 with a set screw and driver down the central lumen 1410 of the counter tensioner 1304.

At 2140, the method may continue by guiding the cord 108 through the lumen 1612 of the tension indicator 2020. The method may continue at 2150 by guiding the cord 108 through the cord receiving nose 1602 of the tensioner 1302, winding the cord 108 around upper cord alignment grooves 1624 of the cord wheel 1604, winding the cord 108 through cord retention notch 1606 and threading the cord 108 through the aperture of central post 1628 of the tensioner 1302. And finally, at 2160, the method of loading the cord on the instruments according to an aspect of the disclosure may include gently tugging or pulling on the loose end of the cord 108 to remove any slack in the cord 108, thus coupling the tensioner 1302 to the tension indicator 2020 and the counter tensioner 1304. At this point, the cord is ready to be tensioned. It will be appreciated that method 2100 may be conducted in a different sequence. Likewise, the method may include the addition of the tensioner extension or may not include the tension indicator, based upon a surgeon’s determination of the appropriate surgical techniques and steps.

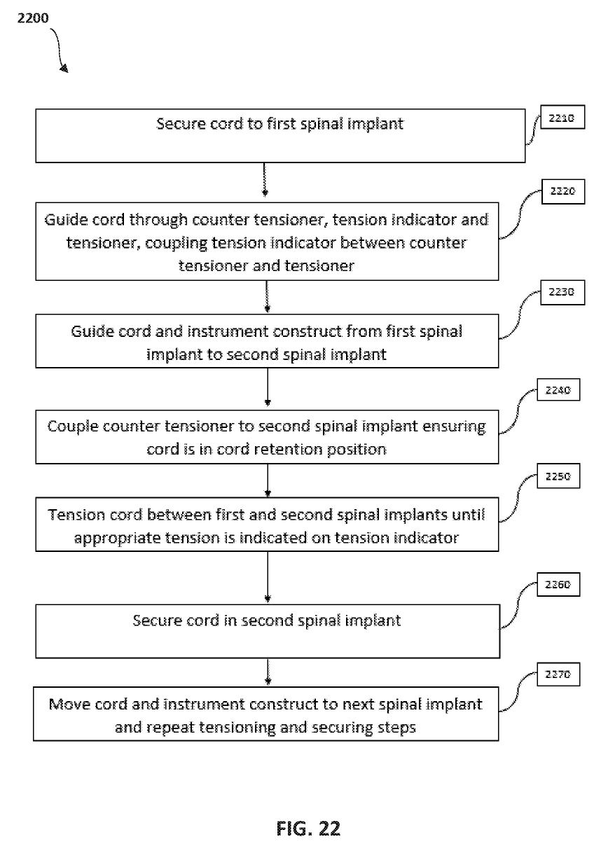

FIG. 22 is a flowchart illustrating an exemplary surgical procedure 2200 for tensioning a surgical cord in a spinal construct utilizing the tensioner 1302, tension indicator 2020 and counter tensioner 1304, according to various aspects of the disclosure is discussed herein below. The 2200 method can begin with the surgeon securing a cord (tether) to a first spinal implant 2210. In an example, the surgeon may secure cord 108 to a first spinal implant 1306 with a set screw (not shown). The surgical procedure may also include the surgeon guiding a cord through a counter tensioner, tension indicator and tensioner, coupling the tension indicator between the counter tensioner and the tensioner 2220. In an example, the surgical procedure may include the surgeon guiding cord 108 from the spinal implant 1306 through port 1318 of counter tensioner 1304; guiding the cord 108 through the lumen 1612 of the tensioner indicator 2020, ensuring the narrow port mating end 2002 is directed toward the counter tensioner port 1318; guiding the cord 108 through the cord receiving nose 1602, around the cord alignment grooves 1624 of the cord wheel 1604, through a cord retention notch 1606 of the cord wheel 1604 and through the central hole of the wheel post 1628, coupling the tension indicator between the counter tensioner 1304 and the tensioner 1302.

In other examples, the surgeon may decide to utilize a tensioner extension and guide the cord 108 through the tensioner extension 810, the tension indicator 2020 and the tensioner 1302. In other examples, a surgeon may decide not to utilize a tension indicator and may guide cord 108 through the counter tensioner 1304 and directly into the tensioner 1302. Alternatively, a surgeon may decide to utilize a tensioner extension and forgo utilizing a tension indicator and guide the cord 108 through the tensioner extension 810 and then through the tensioner 1302.

The surgical procedure may continue with the surgeon guiding the cord and instrument construct of step 2220 from the first spinal implant to a second spinal implant 2230. In an example, the surgeon may move the counter tensioner 1304, tension indicator 2020 and tensioner 1302 instrument construct from the first spinal implant 1306 to an adjacent or nearby spinal implant 1306. The surgical procedure may continue with the surgeon coupling the counter tensioner to the second spinal implant, ensuring that the cord is in the cord retention position within the second spinal implant 2240. In an example of this step, the surgeon may utilize the cord channel 1572 of the counter tensioner 1304 to assist in inserting the cord 108 into the cord channel (not shown) of the second spinal implant 1306. In this step, the surgeon can seat the distal end 1414 of the counter tensioner 1304 on the head of the second spinal implant ensuring the mushroom pins 1572 engage the corresponding engagement features of second spinal implant 1306 and turn the lock knob 1436 at the proximal end 1416 of the counter tensioner to rigidly attach the counter tensioner 1304 to the second implant 1306.

At 2250, the surgical procedure may continue with the surgeon tensioning the cord between the first and second spinal implants until the appropriate tension is indicated on the tension indicator. In an example, tensioning occurs through the surgeon articulating first and second handles 1608 and 1610 of the tensioner 1302, which causes the cord wheel 1604 to rotate and the cord 108 to be tensioned, while counter tension is carried through the counter tensioner 1304 to the second spinal implant 1306 (see FIG. 13A). The tension indicator 2020 between the tensioner 1302 and the counter tensioner 1304 will display the tension. When the desired amount of tension has been applied, the surgical procedure can continue at 2260 with the surgeon securing the cord in the second spinal implant. In an example, the cord 108 may be secured to the second spinal implant 1306 by the surgeon inserting a driver with a set screw down the central lumen 1410 of the counter tensioner 1304 that is secured to the second spinal implant and threading the set screw into the head of the second spinal implant 1306.

At 2270, the surgery may be continued by moving the cord and instrument construct to the next or other nearby spinal implant and repeating the coupling 2240, tensioning 2250 and securing 2260 steps until the appropriate spinal construct and tension is completed.

It will be appreciated that alternative surgical procedure methods may be used by a surgeon. For example, a surgeon may decide to lightly secure the cord to part or all of the surgical construct and then tension and finally tighten the set screws to create the same tension or different tensions across part or all of the surgical construct. Alternatively, as surgeon may decide to forgo utilization of the tension indicator in areas of the construct is minimal tensioning or to utilize tactile tension indications rather than the tension indicator. Additionally, a surgeon may decide to utilize one or more tensioner extensions within the instrumentation. Such surgical decisions do not deter from the concepts discuss herein.

Each of the above non-limiting examples can stand on its own or can be combined in various permutations or combinations with one or more of the other examples.

Additional examples of the presently described method, system, and instrument examples include the following, non-limiting configurations. Each of the following non-limiting examples may stand on its own, or may be combined in any permutation or combination with any one or more of the other examples provided below or throughout the present disclosure.

Example 1 describes subject matter including a system for manipulating implants coupled by a cord. The system may include a tensioner comprising a nose assembly with a cylindrical barrel with a central lumen for receiving the cord into the tensioner; a wheel having one or more cord retention notches and cord alignment grooves on an outer perimeter of the wheel; and two handles for turning the wheel to rotate the cord around the cord alignment grooves of the wheel to tension the cord; and a counter tensioner to guide the cord from the tensioner to an implant. The counter tensioner can comprise an elongate body with a proximal end and a distal end. The proximal end can include a port to receive the nose assembly of the tensioner. The distal end can be releasably couplable to the head of the implant enabling translation of the implant relative to another implant implanted in an adjacent or nearby vertebrae. The counter tensioner can comprise engagement features proximate to the distal end. The engagement features can be configured to maintain an engaged position with cooperating features on the head of the implant when the counter tensioner is in a locked position, maintaining the counter tensioner in an attached position relative to the head of the implant. The engagement features of the counter tensioner can be configured to readily flex out of the way and disengage from the cooperating features on the head of the implant when the counter tensioner is in an unlocked position, enabling the counter tensioner to disengage from the implant.

In example 2, the subject matter of example 1 can include the counter tensioner having a lumen disposed within the elongate body and extending from the proximal end to the distal end thereof, and a set screw engageable with the head of the implant to fix the cord to the implant is deliverable through the lumen of the counter tensioner.

In example 3, the subject matter of example 2 can include the port disposed at an angle that is not parallel to a longitudinal axis of the lumen.

In example 4, the subject matter of any one of examples 1 to 3 can include the counter tensioner including an inner sleeve and an outer sleeve. The inner sleeve can include a plurality of living hinges with the engagement features at a distal end thereof that are engageable with cooperating features on the head of the implant when the outer sleeve is translated distally over the inner sleeve and at least partially over the head of the implant to urge the plurality of engagement features of the living hinges into secure engagement with the cooperating features of the head of the implant.

In example 5, the subject matter of example 4 can include the counter tensioner including a mechanism to prevent the outer sleeve from sliding into a final engaged position if the engagement features of the living hinges are not fully engaged with the cooperating features of the implant head.

In example 6, the subject matter of example 5 can include a feature on the inner sleeve prevents the outer sleeve from moving into a final engaged position if the engagement features of the living hinges are not fully engaged with the cooperating features of the implant head.

In example 7, the subject matter of example 6 can include the cooperating features of the implant head including engagement features that engage the engagement features of the living hinges when engaged.

In example 8, the subject matter of example 7 can include the counter tensioner with a lock knob at the proximal end. The lock knob can be configured to turn to move the out sleeve from a locked to an unlocked position relative to the inner sleeve, causing the engagement features to be in a locked position when the lock knob is in the locked position and the engagement features to be in a flexible position, permitting the engagement features to flex out of the way when the lock knob is in the unlocked position.

In example 9, the subject matter of example 8 can include the counter tensioner including a visual indication proximate to the proximal end that the counter tensioner is in the locked or unlocked position.

In example 10, the subject matter of any one of examples 1 to 9 can include the tensioner including a first handle and a second handle. The nose assembly and cord tensioner wheel are connected to the first handle. The cord wheel can include teeth. The second handle can include complementary teeth features that engage the cord wheel teeth. The second handle can be moveably attached to the first handle. The cord can be tensioned when the second handle is moved relative to the first handle, causing the teeth features of the second handle to engage the teeth connected to the cord tensioner wheel and causing the cord tensioner wheel to rotate and the cord to be tensioned.

In example 11, the subject matter of any one of examples 1 to 10 can include a tension indicator. The tension indicator can include a spring within a housing with a lumen therethrough for receiving the cord. The housing can include a first end with a port for engaging the nose assembly of the tensioner. The tension indicator housing can include a second end with a second port for engaging the port of the counter tensioner. The tensioner indicator can be configured to display the cord tension when the tension indicator is engaged between the tensioner and the counter tensioner.

In example 12, the subject matter of any one of examples 1 to 11 can include a tensioner extension that can include an elongate body with a bore at a proximal end and a nose member at a distal end. The tensioner extension bore at the proximal end can be adapted to receive the nose assembly of the tensioner and the nose member at the distal end can be configured to abut the head of implant that is adapted to receive the cord. The tensioner extension can include a flexible cylindrical member to carry tension along a length of the elongate body. The flexible cylindrical member can be sized to receive the cord through a lumen extending the length of the elongate body.

In example 13, the subject matter of example 12 can include a tension indicator. The tension indicator can include a spring within a housing with a lumen therethrough for receiving the cord. The housing can include a first end with a port for engaging the nose assembly of the tensioner. The tension indicator housing can have a second end with a second port for engaging the tensioner extension bore. The tension indicator can be engaged between the tensioner and the tensioner extension. The tension indicator can be configured to display the cord tension.

Example 14 describes subject matter including a device for tensioning a cord coupled to an implant. The device can include a tensioner including a nose assembly. The nose assembly can include a cylindrical barrel with a central lumen for receiving the cord into the tensioner. The device can include a wheel having one or more cord retention notches and cord alignment grooves on an outer perimeter of the wheel and two handles configured to cause the wheel to rotate, causing the cord to wind around the alignment grooves of the wheel to tension the cord.

In example 15, the subject matter of example 14 can include the tensioner configured to engage a counter tensioner connected to a cord receiving feature on a head of the implant.

In example 16, the subject matter of example 15 can include a tension indicator having a first engagement port and a second engagement port. The tension indicator can be configured to engage the nose assembly of the tensioner with the first engagement port and a port of the counter tensioner with the second engagement port. The tension indicator can display the cord tension at a display area of the tension indicator when the tensioner applies tension to the cord.

In example 17, the subject matter of any of examples 15 or 16 can include the counter tensioner having an inner sleeve with a lumen for receiving the cord and an outer sleeve. The inner sleeve can include living hinges having features that can be configured to flex to engage or disengage complementary features on the implant when the outer sleeve is in an unlocked position and remains engaged with the complementary features on the implant head when the outer sleeve is in a locked position with the inner sleeve.

In example 18, the subject matter of example 17 can include the outer sleeve of the counter tensioner can be configured to remain in an unlocked position if the features of the living hinges of the inner sleeve are not fully engaged with the complementary features of the implant head.

Example 19 describes subject matter including a device for manipulating implants coupled by a cord. The device can include a counter tensioner to guide the cord from a head of an implant to a tensioner. The counter tensioner can include an elongate body having a proximal end and a distal end. The elongate body can include an outer sleeve and an inner sleeve having a lumen configured to receive the cord. The sleeve can include the inner sleeve having more than one living hinge in proximity to the distal end. Each living hinge can have one or more engagement members configured to securely engage with cooperating features on the head of the implant when the inner sleeve is translated upward in the outer sleeve, such that the distal end of the outer sleeve holds the living hinges in place. The counter tensioner can include a knob at the proximal end configured to cause the inner sleeve to translate upwards relative to the outer sleeve. The counter tensioner can include a port at the proximal positioned at an angle from the axis of the elongate body, the port configured to receive a nose assembly of the tensioner.

In example 20, the subject matter of example 19 can include at least one of a locked and an unlocked indication adjacent a window in the outer sleeve near the proximal end of the counter tensioner, such that when the inner sleeve is in a locked position or an unlocked position, a corresponding marking on the inner sleeve aligns with the appropriate indication on the outer sleeve through the window in the outer sleeve.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b) at the time of filing this application, to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for manipulating implants coupled by a cord, the system comprising:
a tensioner comprising:
a nose assembly with a cylindrical barrel with a central lumen for receiving the cord into the tensioner;
a wheel having one or more cord retention notches and cord alignment grooves on an outer perimeter of the wheel; and
two handles for turning the wheel to rotate the cord around the cord alignment grooves of the wheel to tension the cord; and
a counter tensioner to guide the cord from the tensioner to an implant, the counter tensioner comprising an elongate body with a proximal end and a distal end;
wherein the proximal end includes a port to receive the nose assembly of the tensioner;
wherein the distal end is releasably couplable to a head of the implant enabling translation of the implant relative to another implant secured in an adjacent or nearby vertebrae;
wherein the counter tensioner comprises engagement features proximate to the distal end, the engagement features configured to maintain an engaged position with cooperating features on the head of the implant when the counter tensioner is in a locked position, maintaining the counter tensioner in an attached position relative to the head of the implant; and
wherein the engagement features of the counter tensioner are configured to readily flex out of the way and disengage from the cooperating features; on the head of the implant when the counter tensioner is in an unlocked position, enabling the counter tensioner to disengage from the implant.

2. The system of claim 1, wherein the counter tensioner has a lumen disposed within the elongate body and extending from the proximal end to the distal end thereof, and wherein a set screw engageable with the head of the implant to fix the cord to the implant is deliverable through the lumen of the counter tensioner.

3. The system of claim 2, wherein the port is disposed at an angle that is not parallel to a longitudinal axis of the lumen.

4. The system of claim 1, wherein the counter tensioner comprises:
an inner sleeve; and
an outer sleeve;
wherein the inner sleeve comprises a plurality of living hinges with the engagement features at a distal end thereof that are engageable with cooperating features on the head of the implant when the outer sleeve is translated distally over the inner sleeve and at least partially over the head of the implant to urge the plurality of engagement features of the plurality of living hinges into secure engagement with the cooperating features of the head of the implant.

5. The system of claim 4, wherein an inner diameter of a portion of the outer sleeve is prevented from sliding over an outer diameter of a portion of the inner sleeve and into a final engaged position if the engagement features of the plurality of living hinges are not fully engaged with the cooperating features of the head of the implant.

6. The system of claim 5, wherein the plurality of living hinges extend beyond the outer diameter if the engagement features of the plurality of living hinges are not fully engaged with the cooperating features of the head of the implant.

7. The system of claim 4, wherein the cooperating features of the head of the implant comprise female features that engage male engagement features of the plurality of living hinges when engaged.

8. The system of claim 1, wherein the counter tensioner further comprises:
a locking extension extending along a locking extension channel formed in the counter tensioner;
a rotatable pivot lock disposed proximately to the distal end of the counter tensioner; and
a lock handle positioned adjacently to the proximal end of the counter tensioner and configured to rotate between a locked position and an unlocked position;
wherein, when the lock knob is in the locked position, the locking extension is translated toward the pivot lock to prevent rotation of the pivot lock; and
wherein, when the lock knob is in the unlocked position, the locking extension is translated away from the pivot lock to allow rotation of the pivot lock.

9. The system of claim 8, wherein the counter tensioner further comprises a visual indicator proximate to the proximal end and configured to indicate that the counter tensioner is in the locked or unlocked position.

10. The system of claim 1, wherein the tensioner comprises:
a first handle; and
a second handle;
wherein the nose assembly and the wheel are connected to the first handle;
wherein the wheel comprises teeth;
wherein the second handle comprises teeth features complementary to and configured to engage the teeth;
wherein the second handle is moveably attached to the first handle;
wherein, in response to the second handle moving relative to the first handle, the teeth features of the second handle engage the teeth of the wheel to cause the wheel to rotate; and
wherein the cord is tensioned in response to the rotation of the wheel.

11. The system of claim 1 further comprising a tension indicator comprising a spring within a housing with a lumen therethrough for receiving the cord;
wherein the housing further has a first end with a port for engaging the nose assembly of the tensioner;
wherein the tension indicator housing further has a second end with a second port for engaging the port of the counter tensioner; and
wherein, in response to the tension indicator being engaged between the tensioner and the counter tensioner, the tension indicator is configured to display the cord tension.

12. The system of claim 1 further comprising a tensioner extension comprising an elongate body having a bore at a proximal end and a nose member at a distal end;
wherein the bore of the tensioner extension is adapted to receive the nose assembly of the tensioner;
wherein the nose member at the distal end is configured to abut the head of the implant that is adapted to receive the cord;
wherein the tensioner extension comprises a flexible cylindrical member to carry tension along a length of the elongate body; and
wherein the flexible cylindrical member is sized to receive the cord through a lumen extending the length of the elongate body.

13. The system of claim 12 further comprising a tension indicator comprising:
a housing having:
a first end with a first port for engaging the nose assembly of the tensioner; and
a second end with a second port for engaging the tensioner extension bore;
wherein the housing has a lumen extending therethrough between the first and second ends for receiving the cord;
a spring positioned within the housing;
wherein, when the tension indicator is engaged between the tensioner and the tensioner extension, the tension indicator is configured to display the cord tension.

14. A device for tensioning a cord coupled to an implant, the device comprising:
a tensioner comprising a nose assembly, the nose assembly comprising a cylindrical barrel with a central lumen for receiving the cord into the tensioner;
a wheel having one or more cord retention notches and cord alignment grooves on an outer perimeter of the wheel; and
two handles configured to cause the wheel to rotate and the cord causing the cord to wind around the cord alignment grooves of the wheel to tension the cord.

15. The device of claim 14, wherein the tensioner is configured to engage a counter tensioner connected to a cord receiving feature on a head of the implant.

16. The device of claim 15, further comprising a tension indicator having a first engagement port and a second engagement port;
wherein the tension indicator is configured to engage the nose assembly of the tensioner with the first engagement port and a port of the counter tensioner with the second engagement port, wherein, when the tensioner is applies tension to the cord; and
wherein the tension indicator displays the cord tension at a display area of the tension indicator.

17. The device of claim 15, wherein the counter tensioner comprises:
an inner sleeve with a lumen for receiving the cord; and
an outer sleeve;
wherein the inner sleeve comprises living hinges having features that are capable of moving to engage or disengage complementary features on the implant when the outer sleeve is in an unlocked position and remaining engaged with the complementary features on the implant head when the outer sleeve is in a locked position with the inner sleeve.

18. The device of claim 17, wherein the outer sleeve of the counter tensioner is configured to remain in an unlocked position if the features of the living hinges of the inner sleeve are not fully engaged in the complementary features of the implant head.

19. A device for manipulating implants coupled by a cord, the device comprising:
a counter tensioner to guide the cord from a head of an implant to a tensioner, the counter tensioner comprising:
an elongate body having a proximal end and a distal end and comprising:
an outer sleeve; and
an inner sleeve having a lumen configured to receive the cord and comprising a plurality of living hinges in proximity to the distal end;
wherein each living hinge of the plurality of living hinges has one or more engagement members configured to securely engage with cooperating features on the head of the implant when the inner sleeve is translated upward in the outer sleeve such that the distal end of the outer sleeve holds the living hinge in place;
a knob at the proximal end configured to cause the inner sleeve to translate upwards relative to the outer sleeve; and
a port at the proximal positioned at an angle from a longitudinal central axis of the elongate body, the port configured to receive a nose assembly of the tensioner.

20. The device of claim 19, further comprising at least one of a locked and an unlocked indication adjacent to a window in the outer sleeve near the proximal end of the counter tensioner such that when the inner sleeve is in a locked position or an unlocked position, a corresponding marking on the inner sleeve aligns with the appropriate indication on the outer sleeve through the window in the outer sleeve.
